(19) United States
(12) Patent Application Publication
(10) Pub. No.: US 2024/0325003 A1
(43) Pub. Date: Oct. 3, 2024

(54) DUCTAL SAMPLING DEVICE

(71) Applicant: GYRUS ACMI, IN D/B/A OLYMPUS SURGICAL TECHNOLOGIES AMERICA, Westborough, MA (US)

(72) Inventors: Anthony R. Pirozzi, Raleigh, NC (US); Kester Julian Batchelor, Mound, MN (US)

(21) Appl. No.: 18/615,393

(22) Filed: Mar. 25, 2024

Related U.S. Application Data
(60) Provisional application No. 63/492,823, filed on Mar. 29, 2023.
(60) Provisional application No. 63/472,481, filed on Jun. 12, 2023.

(51) Int. Cl. A61B 10/04; A61B 10/02; A61B 90/00

(52) U.S. Cl. CPC A61B 10/04; A61B 10/0266; A61B 10/0283; A61B 90/361; A61B 2010/0208; A61B 2090/3612

(57) ABSTRACT

A ductal sampling device comprises an elongate shaft comprising a proximal end portion and a distal end portion comprising a textured scraping body, and a sheath configured to slide over the elongate shaft to alternatively cover and cover the textured scraping body. A method of collecting biological matter using a ductal sampling device comprises inserting the ductal sampling device into anatomy of a patient, guiding a textured scraping body of the ductal sampling device to a target tissue, grating the textured scraping body against the target tissue, and collecting biological matter from the target tissue with the textured scraping body.

### PRIORITY CLAIM

This application claims the benefit of priority to U.S. Provisional Patent Application Ser. No. 63/492,823, filed Mar. 29, 2023, and U.S. Provisional Patent Application Ser. No. 63/472,481, filed Jun. 12, 2023, the contents of which are hereby incorporated by reference in their entireties.

### TECHNICAL FIELD

The present disclosure relates generally to medical devices comprising elongate bodies configured to be inserted into incisions or openings in anatomy of a patient to provide diagnostic or treatment operations. More specifically, the present disclosure relates to tissue retrieval devices, such as biopsy devices, that can be inserted into anatomy of a patient to perform a biological matter removal and collection process, such as by scraping or cutting sample tissue for analysis.

### BACKGROUND

Endoscopes can be used for one or more of 1) providing passage of other devices, e.g., therapeutic devices or tissue collection devices, toward various anatomical portions, and 2) obtaining imaging of such anatomical portions. Such anatomical portions can include gastrointestinal tract (e.g., esophagus, stomach, duodenum, pancreaticobiliary duct, intestines, colon), renal area (e.g., kidney(s), ureter, bladder, urethra) and other internal organs (e.g., reproductive systems, sinus cavities, submucosal regions, respiratory tract), and the like.

Conventional endoscopes can be involved in a variety of clinical procedures, including, for example, illuminating, imaging, detecting and diagnosing one or more disease states, providing fluid delivery (e.g., saline or other preparations via a fluid channel) toward an anatomical region, providing passage (e.g., via a working channel) of one or more therapeutic devices for sampling or treating an anatomical region, and providing suction passageways for collecting fluids (e.g., saline or other preparations) and the like.

In conventional endoscopy, the distal portion of the endoscope can be configured for supporting and orienting a therapeutic device. In some systems, two endoscopes can be configured to work together with a first endoscope guiding a second endoscope inserted therein with the aid of an elevator. Such systems can be helpful in guiding endoscopes to anatomic locations within the body that are difficult to reach. For example, some anatomic locations can only be accessed with an endoscope after insertion through a circuitous path.

### SUMMARY

The present disclosure recognizes that problems to be solved with conventional medical devices, and in particular endoscopes and duodenoscopes used to retrieve sample biological matter from target tissue, include, among other things, 1) the difficulty in navigating endoscopes, and instruments inserted therein, to locations in anatomical regions deep within a patient, 2) the disadvantage of only being able to obtain small tissue sample sizes, 3) the potential for obtaining the wrong sample tissue if the target tissue is not adequately engaged, and 4) the increased time and associated cost of having to repeatedly remove and reinsert medical devices to obtain a sufficient quantity of sample material.

Such problems can be particularly present in duodenoscopy procedures (e.g., Endoscopic Retrograde Cholangio-Pancreatography, hereinafter “ERCP” procedures) where an auxiliary scope (also referred to as daughter scope, or cholangioscope) can be attached and advanced through the working channel of a main scope (also referred to as mother scope or duodenoscope). Furthermore, another device (e.g., a treatment or therapeutic device), such as a tissue retrieval device used for biopsies, can be inserted into the auxiliary scope. As such, the duodenoscope, auxiliary scope and tissue retrieval device become progressively smaller and more difficult to maneuver and perform interventions and treatments.

In the case of ERCP, when a physician wants to get tissue samples, it is desirable to have a device that can pass through the working channel of a cholangioscope. As such, the device is typically less than or equal to 1.2 mm in diameter. Biopsy forceps are typically the only option for sampling tissue during cholangioscopy, i.e., with the cholangioscope. Forceps for use with cholangioscopes are sometimes referred to as baby biopsy forceps due to their small size. Baby biopsy forceps, however, leave several user needs unaddressed. For example, since the cup size of a baby biopsy forceps is limited by the diameter of working channel for cholangioscope, baby biopsy forceps cannot typically obtain enough volume of tissue samples. Users may decide to use a larger sized biopsy forceps without using the cholangioscope. However, the ability to use direct visualization of the forceps using the imaging capabilities of the cholangioscope is lost. In addition to that, when users try to separate the sample from the patient body, there is a case that some samples get left behind, such as by remaining stuck to the forceps cup. There is a desire for more effective ways to capture the tissue samples.

Alternatives to biopsy forces include the cytology brush. However, users are not satisfied with cytology brushes on the current market because, for example, a plethora of studies show the diagnostic yield is inadequate. Specifically, users are not satisfied with the ability of the cytology brush to agitate the region of suspected malignancy as agitation is not done under direct visualization. For example, the bristles of most brushes currently on the market are too soft to obtain sufficient diagnostic material. Thus, there is a desire for a device that is capable of scraping more tissue than existing brushes. Additionally, cytology brushes are typically not used within a cholangioscope due to their large size and are instead used within the duodenoscope. In examples, cytology brushes can be used with guidewires. In addition to that, users are not satisfied with the ability of cytology brushes to trap the tissue. For example, collected tissue can become dislodged from the bristles of the bushes before being collected for analysis. As such, there is also a desire of capability of capturing the tissue or preventing the tissue from falling out of the cytology brush.

The present disclosure can help provide solutions to these and other problems by providing systems, devices and methods relating to tissue retrieval devices, such as ductal sampling devices that can have textured scraping bodies for dragging along the wall of an anatomic duct. The textured scraping bodies can be shaped to have cutting elements and storage spaces adjacent the cutting elements. The cutting elements can be configured to detach target tissue from the anatomy and the storage spaces can be configured to retain the detached target tissue. The textured scraping bodies can have small-diameters and relatively long lengths. The small diameter of such textured scraping bodies can allow for the tissue retrieval devices to be used within the working channel of an auxiliary scope, such as a cholangioscope, to take advantage of imaging capabilities of the auxiliary scope. The length of the textured scraping body can increase the ability of the tissue retrieval device to engage the correct target tissue. Furthermore, the length of the textured scraping body can allow for the tissue retrieval devices to obtain many small samples that increase the overall sample volume that can be obtained, thereby reducing, or eliminating the need to remove the endoscope from the anatomy to perform multiple tissue retrieval iterations. The textured scraping body can be selectively covered by a user-operated sheath to prevent damaging the auxiliary scope and tissue proximate the target tissue and to prevent collected target tissue from being lost during retrieval of the ductal sampling device.

As such, the present disclosure can help solve the problems referenced above and other problems by 1) being compatible with auxiliary scopes and imaging systems thereof, 2) increasing the capacity of sample material collected with each insertion, 3) increasing the likelihood of the tissue retrieval device of engaging the intended target tissue, 4) reducing the number of times a tissue retrieval device needs to be inserted and reinserted into the anatomy, and 5) reducing the likelihood that collected sample tissue is lost during retrieval of the endoscope, as is described herein, such as by using an elongate, small-diameter and flexible textured scraping body that can be covered an uncovered with a sheath.

In an example, a ductal sampling device can comprise an elongate shaft comprising a proximal end portion and a distal end portion comprising a textured scraping body, and a sheath configured to slide over the elongate shaft to alternatively cover and cover the textured scraping body.

In another example, a method of collecting biological matter using a ductal sampling device can comprise inserting the ductal sampling device into anatomy of a patient, guiding a textured scraping body of the ductal sampling device to a target tissue, grating the textured scraping body against the target tissue, and collecting biological matter from the target tissue with the textured scraping body.

### BRIEF DESCRIPTION OF DRAWINGS

FIG. 1 is a schematic diagram of an endoscopy system comprising an imaging and control system and an endoscope, such as duodenoscope, with which the ductal sampling devices of the present disclosure can be used.

FIG. 2 is a schematic diagram of the imaging and control system of FIG. 1 showing the imaging and control system connected to the endoscope.

FIG. 3A is a schematic top view of a distal portion of the endoscope of FIG. 2 comprising a camera module including optical components for a side-viewing endoscope and an elevator mechanism.

FIG. 3B is a cross-sectional view taken along the plane 3B-3B of FIG. 3A showing the optical components.

FIG. 3C is a cross-sectional view taken along the plane 3C-3C of FIG. 3A showing the elevator mechanism.

FIG. 4A is an end view of a camera module including optical and functional components suitable for use as an auxiliary scope that can be used with the endoscope of FIGS. 1 and 2.

FIG. 4B is a cross-sectional view taken along section 4B-4B of FIG. 4A showing components of the camera module arranged in an end-viewing configuration.

FIG. 5 is a schematic illustration of a distal portion of an endoscope being used to position an auxiliary scope proximate a duodenum, the auxiliary scope being configured to receive a ductal sampling device of the present disclosure.

FIG. 6 is a side view of a ductal sampling device of the present disclosure comprising a textured scraping body.

FIG. 7 is a close-up view of an inner catheter that connects to the textured scraping body of FIG. 6 extending from an outer sheath.

FIG. 8 is a close-up view of a distal end of the textured scraping body of FIG. 6 showing an atraumatic tip.

FIG. 9 is a close-up view of the textured scraping body of FIG. 6 comprising cutting elements and storage spaces.

FIG. 10 side cross-sectional view of the textured scraping body of FIG. 9 showing an internal passage.

FIG. 11A is a cross-sectional view through the textured scraping body of FIG. 10 showing a sheath covering the shaft and an internal passage within the shaft.

FIG. 11B is a cross-sectional view through the textured scraping body of FIG. 10 showing teeth.

FIG. 11C is a cross-sectional view through the textured scraping body of FIG. 10 showing pockets.

FIG. 12 is a schematic side view of the ductal sampling device of FIG. 6 showing the textured scraping body connected to the shaft via a break-away section.

FIG. 13 is a perspective view of a shaft of a ductal sampling device of the present disclosure comprising a slit.

FIG. 14 is a cross-sectional view of the shaft of FIG. 13 having a scraper paddle extending into the slit.

FIG. 15 is a schematic side view of opposing edges of a textured scraping body of the present disclosure comprising a cutting edge opposing a sliding edge.

FIG. 16 is a schematic side view of opposing edges of a textured scraping body of the present disclosure comprising a straight cutting edge opposing an angled cutting edge.

FIG. 17 is a perspective view of a textured scraping body of the present disclosure comprising a flexible section including flexible links.

FIG. 18A is a top view of the flexible links in an expanded state at rest.

FIG. 18B is a top view of the flexible links of FIG. 18A in a contracted state under compression.

FIG. 18C is a schematic side view of the flexible links of FIG. 17 incorporating scraping edges.

FIG. 19A is a schematic side view of a textured scraping body for a ductal sampling device of the present disclosure including an activation wire.

FIG. 19B is a schematic side view of the textured scraping body of FIG. 19A with the activation wire pulled to induce flexing of the ductal sampling device.

FIG. 19C is a schematic side view of the textured scraping body of FIG. 19A with the activation wire pulled to induce bulging of the ductal sampling device.

FIG. 20A is a schematic side view of a textured scraping body for a ductal sampling device of the present disclosure comprising enlarged pockets forming scraping edges aligned along the ductal sampling device to face in a single direction.

FIG. 20B is a cross-sectional view through one of the enlarged pockets of FIG. 20A showing a trough and a sidewall of the enlarged pocket.

FIG. 20C is a top view of one of the enlarged pockets of FIG. 20A showing scraping edges overhanging a trough.

FIG. 21 is a schematic side view of a textured scraping body for a ductal sampling device of the present disclosure comprising enlarged pockets forming scraping edges arranged in an alternating pattern to face in opposite directions.

FIG. 22 is a perspective view of a textured scraping body of the present disclosure comprising outwardly facing scraping tabs.

FIG. 23 is a perspective view of a sheath for use with the textured scraping body of FIG. 22 having standoffs to provide separation from the outwardly facing scraping tabs.

FIG. 24 is a schematic cross-sectional view of a ductal sampling device of the present disclosure connected to a fluid injection and suction system.

FIG. 25 is block diagram illustrating methods of collecting biological matter from a patient using the tissue retrieval devices of the present disclosure, such as a ductal sampling device having a textured scraping body.

### DETAILED DESCRIPTION

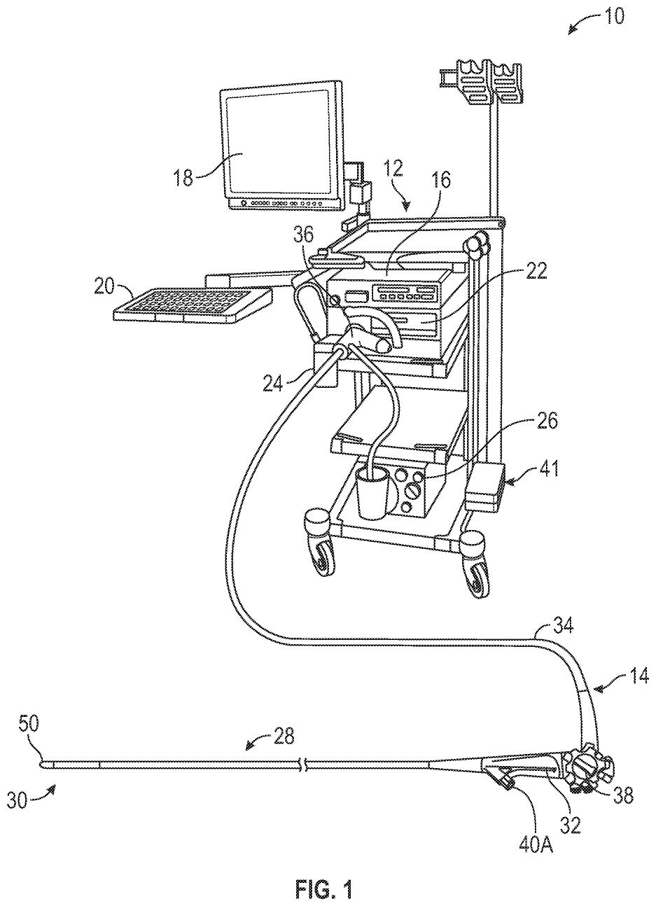

FIG. 1 is a schematic diagram of endoscopy system 10 comprising imaging and control system 12 and endoscope 14. The system of FIG. 1 is an illustrative example of an endoscopy system suitable for use with the systems, devices and methods described herein, such as tissue collection, retrieval and storage devices and biopsy instruments that can be used for obtaining samples of tissue or other biological matter to be removed from a patient for analysis or treatment of the patient, including ductal sampling devices having textured scraping bodies. According to some examples, endoscope 14 can be insertable into an anatomical region for imaging and/or to provide passage of one or more sampling devices for biopsies, or one or more therapeutic devices for treatment of a disease state associated with the anatomical region. Endoscope 14 can, in advantageous aspects, interface with and connect to imaging and control system 12. In the illustrated example, endoscope 14 comprises a duodenoscope, though other types of endoscopes can be used with the features and teachings of the present disclosure.

Imaging and control system 12 can comprise control unit 16, output unit 18, input unit 20, light source unit 22, fluid source 24 and suction pump 26.

Imaging and control system 12 can include various ports for coupling with endoscopy system 10. For example, control unit 16 can include a data input/output port for receiving data from and communicating data to endoscope 14. Light source unit 22 can include an output port for transmitting light to endoscope 14, such as via a fiber optic link. Fluid source 24 can include a port for transmitting fluid to endoscope 14. Fluid source 24 can comprise a pump and a tank of fluid or can be connected to an external tank, vessel or storage unit. Suction pump 26 can comprise a port used to draw a vacuum from endoscope 14 to generate suction, such as for withdrawing fluid from the anatomical region into which endoscope 14 is inserted. Output unit 18 and input unit 20 can be used by an operator of endoscopy system 10 to control functions of endoscopy system 10 and view output of endoscope 14. Control unit 16 can additionally be used to generate signals or other outputs from treating the anatomical region into which endoscope 14 is inserted. In examples, control unit 16 can generate electrical output, acoustic output, a fluid output and the like for treating the anatomical region with, for example, cauterizing, cutting, freezing and the like.

Endoscope 14 can comprise insertion section 28, functional section 30 and handle section 32, which can be coupled to cable section 34 and coupler section 36.

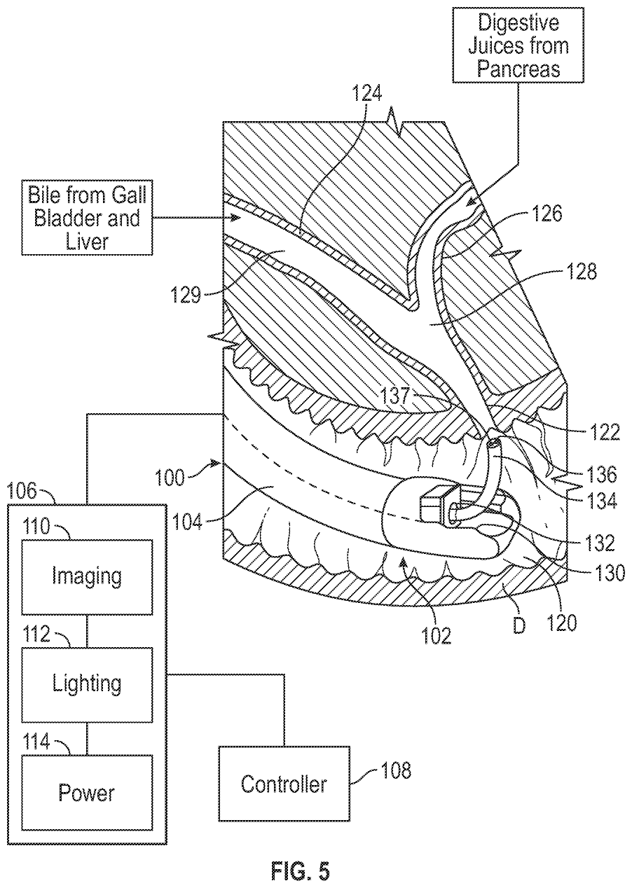

Insertion section 28 can extend distally from handle section 32 and cable section 34 can extend proximally from handle section 32. Insertion section 28 can be elongate and include a bending section, and a distal end to which functional section 30 can be attached. The bending section can be controllable (e.g., by control knob 38 on handle section 32) to maneuver the distal end through tortuous anatomical passageways (e.g., stomach, duodenum, kidney, ureter, etc.). Insertion section 28 can also include one or more working channels (e.g., an internal lumen) that can be elongate and support insertion of one or more therapeutic tools of functional section 30, such as auxiliary scope 134 of FIG. 5. The working channel can extend between handle section 32 and functional section 30. Additional functionalities, such as fluid passages, guide wires, and pull wires can also be provided by insertion section 28 (e.g., via suction or irrigation passageways, and the like).

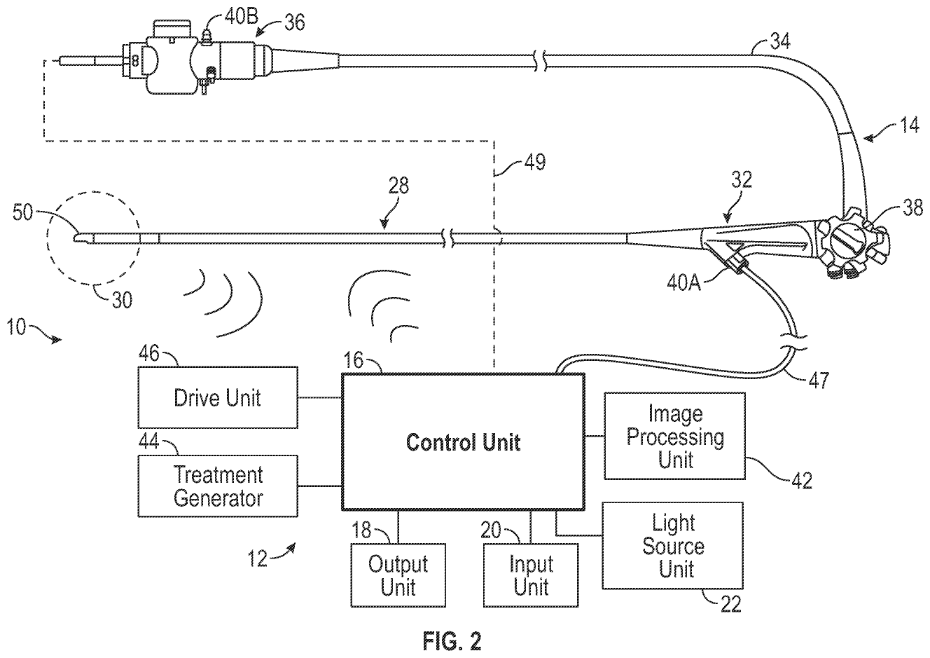

Handle section 32 can comprise control knob 38 as well as port 40A and port 40B (FIG. 2). Control knob 38 can be coupled to a pull wire, or other actuation mechanisms, extending through insertion section 28. Port 40A, as well as other ports, such as port 40B (FIG. 2) can be configured to couple various electrical cables, guide wires, auxiliary scopes, tissue collection devices of the present disclosure, fluid tubes and the like to handle section 32 for coupling with insertion section 28. In examples, port 40A can be used to feed an auxiliary scope, cholangioscope or ductal sampling device into insertion section 28. For example, a ductal sampling device of the present disclosure can be directly inserted into port 40A or can be inserted into a cholangioscope inserted into port 40A.

Imaging and control system 12, according to examples, can be provided on a mobile platform (e.g., cart 41) with shelves for housing light source unit 22, suction pump 26, image processing unit 42 (FIG. 2), etc. Alternatively, several components of imaging and control system 12 shown in FIGS. 1 and 2 can be provided directly on endoscope 14 so as to make the endoscope “self-contained.”

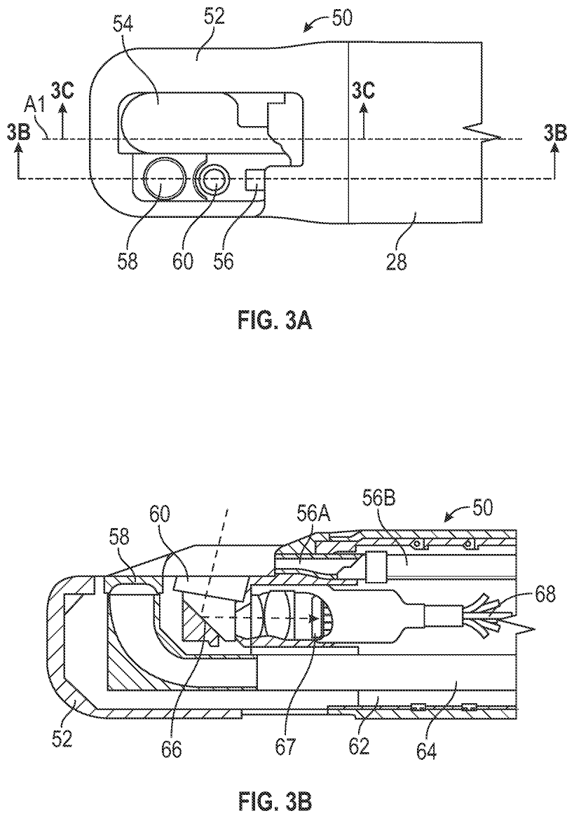

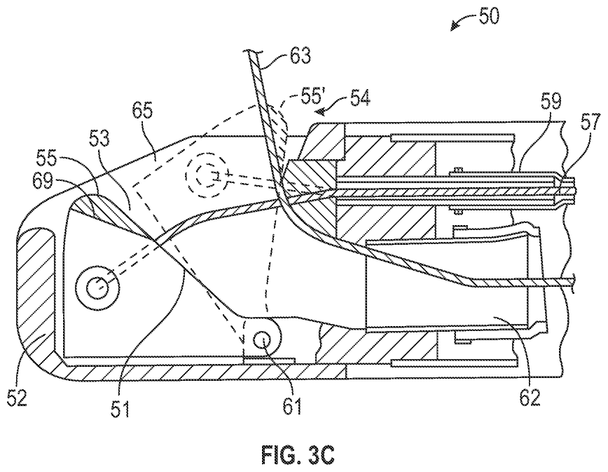

Functional section 30 can comprise components for treating and diagnosing anatomy of a patient. Functional section 30 can comprise an imaging device, an illumination device and an elevator, such as is described further with reference to elevator 54 of FIGS. 3A-3C. Functional section 30 can further comprise or be used with biological matter and tissue collection and retrieval devices as are described herein, such as a ductal sampling device having a textured scraping body. Operation of some or all features of functional section 30 is typically performed at imaging and control system 12.

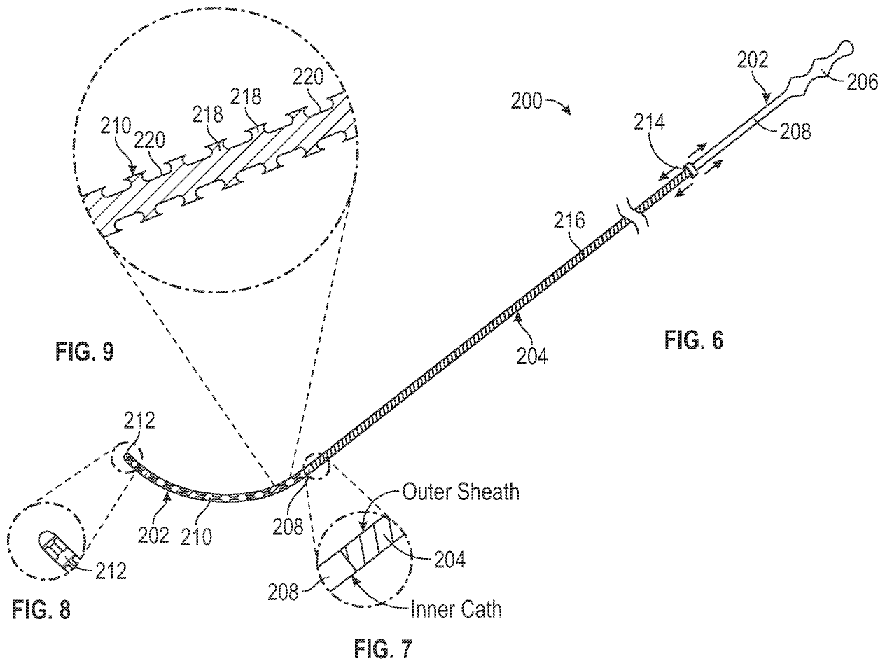

FIG. 2 is a schematic diagram of endoscopy system 10 of FIG. 1 comprising imaging and control system 12 and endoscope 14. FIG. 2 schematically illustrates components of imaging and control system 12 coupled to endoscope 14, which in the illustrated example comprises a duodenoscope. Imaging and control system 12 can comprise control unit 16, which can include or be coupled to image processing unit 42, treatment generator 44 and drive unit 46, as well as light source unit 22, input unit 20 and output unit 18. In examples, control unit 16 be in communication with or coupled to ductal sampling device 200 (FIG. 6), which can comprise a device configured to engage tissue and collect and store a portion of that tissue.

Coupler section 36 can be connected to control unit 16 via cable 49 (shown schematically in FIG. 2) to connect to endoscope 14 to multiple features of control unit 16, such as image processing unit 42 and treatment generator 44. In examples, port 40A can be used to insert another instrument or device, such as a daughter scope, auxiliary scope and/or a ductal sampling device, into endoscope 14. Such instruments and devices can be independently connected to control unit 16 via cable 47. In examples, port 40B can be used to connect coupler section 36 to various inputs and outputs, such as video, air, light and electric. Control unit 16 can be configured to activate a camera to view target tissue distal of ductal sampling device 200 (FIG. 6) when ductal sampling device 200 is positioned to extend from insertion section 28. Likewise, control unit 16 can be configured to activate light source unit 22 to shine light on ductal sampling device 200.

Image processing unit 42 and light source unit 22 can each interface with endoscope 14 (e.g., at functional section 30) by wired or wireless electrical connections. Imaging and control system 12 can accordingly illuminate an anatomical region, collect signals representing the anatomical region, process signals representing the anatomical region, and display images representing the anatomical region on output unit 18. Imaging and control system 12 can include light source unit 22 to illuminate the anatomical region using light of desired spectrum (e.g., broadband white light, narrow-band imaging using preferred electromagnetic wavelengths, and the like). Imaging and control system 12 can connect (e.g., via an endoscope connector) to endoscope 14 for signal transmission (e.g., light output from light source, video signals from imaging system in the distal end, diagnostic and sensor signals from a diagnostic device, and the like).

Fluid source 24 (FIG. 1) can be in communication with control unit 16 and can comprise one or more sources of air, saline or other fluids, as well as associated fluid pathways (e.g., air channels, irrigation channels, suction channels) and connectors (barb fittings, fluid seals, valves and the like). Fluid source 24 can be utilized as an activation energy for a biasing device or a pressure-applying device of the present disclosure. Imaging and control system 12 can also include drive unit 46, which can be an optional component. Drive unit 46 can comprise a motorized drive for advancing a distal section of endoscope 14, as described in at least PCT Pub. No. WO 2011/140118 A1 to Frassica et al., titled “Rotate-to-Advance Catheterization System,” which is hereby incorporated in its entirety by this reference.

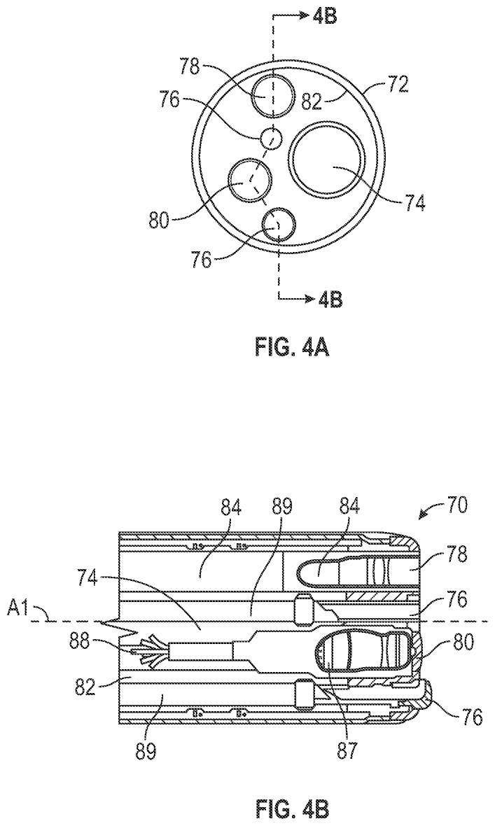

FIGS. 3A-3C illustrate a first example of functional section 30 of endoscope 14 of FIG. 2. FIG. 3A illustrates a top view of functional section 30. FIG. 3B illustrates a cross-sectional view of functional section 30 taken along section plane 3B-3B of FIG. 3A. FIG. 3C illustrates a cross-sectional view of functional section 30 taken along section plane 3C-3C of FIG. 3A. FIGS. 3A-3C illustrate side-viewing endoscope camera module 50, such as can be used with a duodenoscope. In side-viewing endoscope camera module 50, illumination and imaging systems are positioned such that the viewing angle of the imaging system corresponds to a target anatomy lateral to central longitudinal axis A1 of endoscope 14. However, the biological matter retrieval devices and ductal sampling devices of the present disclosure can be used with other types of endoscopes, such as end-viewing endoscopes discussed with reference to FIGS. 4A and 4B.

In the example of FIGS. 3A and 3B, side-viewing endoscope camera module 50 can comprise housing 52, elevator 54, fluid outlet 56, illumination lens 58 and objective lens 60. Housing 52 can form a fluid tight coupling with insertion section 28. Housing 52 can comprise opening for elevator 54. Elevator 54 can comprise a mechanism for moving a device inserted through insertion section 28, such as auxiliary scope 134 of FIG. 5. In particular, elevator 54 can comprise a device that can bend an elongate device extended through insertion section 28 along axis A1, as is discussed in greater detail with reference to FIG. 3C. Elevator 54 can be used to bend the elongate device at an angle to axis A1 to thereby treat or access the anatomical region adjacent side-viewing endoscope camera module 50. Elevator 54 is located alongside, e.g., radially outward of axis A1, illumination lens 58 and objective lens 60.

As can be seen in FIG. 3B, insertion section 28 can comprise central lumen 62 through which various components (e.g., auxiliary scope 134 (FIG. 5) can be extended to connect functional section 30 with handle section 32 (FIG. 2). For example, illumination lens 58 can be connected to light transmitter 64, which can comprise a fiber optic cable or cable bundle extending to light source unit 22 (FIG. 1). Likewise, objective lens 60 can be coupled to prism 66 and image processing unit 67, which can be coupled to wiring 68. Also, fluid outlet 56A can be coupled to fluid line 56B, which can comprise a tube extending to fluid source 24 (FIG. 1). Other elongate elements, e.g., tubes, wires, cables, can extend through central lumen 62 to connect functional section 30 with components of endoscopy system 10, such as suction pump 26 (FIG. 1) and treatment generator 44 (FIG. 2).

FIG. 3C a schematic cross-sectional view taken along section plane 3C-3C of FIG. 3C showing elevator 54. Elevator 54 can comprise deflector 55 that can be disposed in accommodation space 53 of housing 52. Deflector 55 can be connected to wire 57, which can extend through tube 59 to connect to handle section 32. Wire 57 can be actuated, such as by rotating a knob, pulling a lever, or pushing a button on handle section 32. Movement of wire 57 can cause rotation, e.g., clockwise, from a first position of deflector 55 about pin 61 to a second position of deflector 55, indicated by deflector 55′. Deflector 55 can be actuated by wire 57 to move the distal portion of instrument 63 extending through window 65 in housing 52.

Housing 52 can comprise accommodation space 53 that houses deflector 55. Instrument 63 can comprise forceps, a guide wire, a catheter, or the like that extends through central lumen 62. Instrument 63 can additionally comprise auxiliary scope 134 of FIG. 5, or a tissue collection device such as ductal sampling device 200 FIGS. 6-9, as well as other instruments including other biopsy instruments or ductal sampling devices described herein. A proximal end of deflector 55 can be attached to housing 52 at pin 61 provided to side-viewing endoscope camera module 50. A distal end of deflector 55 can be located below window 65 within housing 52 when deflector 55 is in the lowered, or un-actuated, state. The distal end of deflector 55 can at least partially extend out of window 65 when deflector 55 is raised, or actuated, by wire 57. Instrument 63 can slide on angled ramp surface 51 of deflector 55 to initially deflect the distal end of instrument 63 toward window 65. Angled ramp surface 51 can facilitate extension of the distal portion of instrument 63 extending from window 65 at a first angle relative to the axis of central lumen 62. Angled ramp surface 51 can include groove 69, e.g., a v-notch, to receive and guide instrument 63. Deflector 55 can be actuated to bend instrument 63 at a second angle relative to the axis of central lumen 62, which is closer to perpendicular that the first angle. When wire 57 is released, deflector 55 can be rotated, e.g., counter-clockwise, back to the lowered position, either by pushing or relaxing of wire 57. In examples, instrument 63 can comprise a cholangioscope or auxiliary scope 134 (FIG. 5).

Side-viewing endoscope camera module 50 of FIGS. 3A-3C can include optical components (e.g., objective lens 60, prism 66, image processing unit 67, wiring 68) for collection of image signals, lighting components (e.g., illumination lens 58, light transmitter 64) for transmission or generation of light. Side-viewing endoscope camera module 50 can also include a photosensitive element, such as a charge-coupled device (“CCD” sensor) or a complementary metal-oxide semiconductor (“CMOS”) sensor. In either example, image processing unit 67 can be coupled (e.g., via wired or wireless connections) to image processing unit 42 (FIG. 2) to transmit signals from the photosensitive element representing images (e.g., video signals) to image processing unit 42, in turn to be displayed on a display such as output unit 18. In various examples, imaging and control system 12 and image processing unit 67 can be configured to provide outputs at desired resolution (e.g., at least 480p, at least 720p, at least 1080p, at least 4K UHD, etc.) suitable for endoscopy procedures.

Thus, as an endoscope is inserted further into the anatomy, the complexity with which it must be maneuvered and contorted increases, as described with reference to FIG. 5. Furthermore, in order to reach locations even further in the anatomy, additional devices can be used, e.g., instrument 63 in the form of auxiliary scope 134 (FIG. 5). As such, the cross-sectional area, e.g., diameter, of subsequently nested devices becomes smaller, thereby requiring even smaller devices that can be difficult to manufacture and manipulate, or satisfactorily produce results without repeated interventions (e.g., interactions with the patient).

FIG. 4A illustrates an end view of end-viewing endoscope camera module 70 and FIG. 4B illustrates a cross-sectional view of end-viewing endoscope camera module 70 taken along section plane 4B-4B of FIG. 4A. FIGS. 4A and 4B each illustrate end-viewing endoscope camera module 70, such as for use as a gastroscope, colonoscope, cholangioscope, and the like. In end-viewing endoscope camera module 70, illumination and imaging systems are positioned such that the viewing angle of the imaging system corresponds to a target anatomy located adjacent (e.g., distal of) an end of endoscope 14 and in line with a central longitudinal axis of endoscope 14.

End-viewing endoscope camera module 70 of FIGS. 4A and 4B can be used as an alternative example of functional section 30 of endoscope 14 of FIGS. 1 and 2. Additionally, end-viewing endoscope camera module 70 can be used in a cholangioscope, such as auxiliary scope 134 of FIG. 5.

In the example of FIGS. 4A and 4B, end-viewing endoscope camera module 70 can comprise housing 72, therapy unit 74, fluid outlets 76, illumination lens 78 and objective lens 80. Housing 72 can comprise and endcap for insertion section 28, thereby providing a seal to lumen 82.

As can be seen in FIG. 4B, insertion section 28 can comprise lumen 82 through which various components can be extended to connect end-viewing endoscope camera module 70 with handle section 32 (FIG. 2), for example. For example, illumination lens 78 can be connected to light transmitter 84, which can comprise a fiber optic cable or cable bundle extending to light source unit 22 (FIG. 1). Likewise, objective lens 80 can be coupled to imaging unit 87, which can be coupled to wiring 88. Also, fluid outlets 76 can be coupled to fluid lines 89, which can comprise a tube extending to fluid source 24 (FIG. 1). In examples, one of fluid outlets 76 can comprise an inlet connected to a fluid line 89 configured for suction, such as being connected to a vacuum, for recovery of lavage and irrigation fluid. Other elongate elements, e.g., tubes, wires, cables, can extend through lumen 82 to connect functional section 30 with components of endoscopy system 10, such as suction pump 26 (FIG. 1) and treatment generator 44 (FIG. 2). For example, therapy unit 74 can comprise a wide-diameter lumen for receiving other treatment components, such as cutting devices and therapeutic devices including tissue separator devices.

End-viewing endoscope camera module 70 can also include a photosensitive element, such as a charge-coupled device (“CCD” sensor) or a complementary metal-oxide semiconductor (“CMOS”) sensor. In either example, imaging unit 87 can be coupled (e.g., via wired or wireless connections) to image processing unit 42 (FIG. 1) to transmit signals from the photosensitive element representing images (e.g., video signals) to image processing unit 42, in turn to be displayed on a display such as output unit 18. In various examples, imaging and control system 12 and imaging unit 87 can be configured to provide outputs at desired resolution (e.g., at least 480p, at least 720p, at least 1080p, at least 4K UHD, etc.) suitable for endoscopy procedures.

FIG. 5 is a schematic illustration of distal portion of endoscope 100 according to the present disclosure positioned in duodenum D. Endoscope 100 can comprise functional module 102, insertion section module 104, and control module 106. Control module 106 can include controller 108. Control module 106 can include other components, such as those described with reference to endoscopy system 10 (FIG. 1) and control unit 16 (FIG. 2). Additionally, control module 106 can comprise components for controlling a camera and a light source connected to auxiliary scope 134, such as imaging unit 110, lighting unit 112 and power unit 114. Endoscope 100 can be configured similarly as endoscope 14 of FIGS. 1 and 2.

Duodenum D can comprise duct wall 120, sphincter of Oddi 122, common bile duct 124 and main pancreatic duct 126. Duodenum D comprises an upper part of the small intestine. Common bile duct 124 carries bile from the gallbladder and liver (not illustrated) and empties the bile into the duodenum D through sphincter of Oddi 122. Main pancreatic duct 126 carries pancreatic juice from the exocrine pancreas (not illustrated) to common bile duct 124. Sometimes it can be desirable to remove biological matter, e.g., tissue, from common bile duct 124 or main pancreatic duct 126 to analyze the tissue to, for example, diagnose diseases or maladies of the patient such as cancer.

Functional module 102 can comprise elevator portion 130. Endoscope 100 can further comprise lumen 132 and auxiliary scope 134. Auxiliary scope 134 can comprise lumen 136. Auxiliary scope 134 can itself include functional components, such as camera lens 137 and a light lens (not illustrated) coupled to control module 106, to facilitate navigation of auxiliary scope 134 from endoscope 100 through the anatomy and to facilitate viewing of components extending from lumen 132.

In certain duodenoscopy procedures (e.g., Endoscopic Retrograde Cholangio-Pancreatography, hereinafter “ERCP” procedures) an auxiliary scope (also referred to as daughter scope, or cholangioscope), such as auxiliary scope 134, can be attached and advanced through lumen 132 (or central lumen 62 of insertion section 28 of endoscope 14 in FIG. 3B) of the “main scope” (also referred to as mother scope, or duodenoscope), such as endoscope 100. As discussed in greater detail below, auxiliary scope 134 can be guided into sphincter of Oddi 122. Therefrom, a surgeon operating auxiliary scope 134 can navigate auxiliary scope 134 through lumen 132 toward the gall bladder, liver or other locations in the gastrointestinal system to perform various procedures. The surgeon can navigate auxiliary scope 134 past entry 128 of main pancreatic duct 126 and into passage 129 of common bile duct 124, or into entry 128. Auxiliary scope 134 can be used to guide an additional device to the anatomy to obtain biological matter, such as by passage through or attachment to lumen 136. The additional device can have its own functional devices, such as a light source, camera, tissue separators, accessories, and biopsy channel, for therapeutic procedures. As described with reference to FIGS. 6-9, the additional device can include various features, such as ductal sampling device 200 including textured scraping body 210, for gathering biological matter, such as tissue. The biological matter can then be removed from the patient, typically by removal of the additional device from the auxiliary device, so that the removed biological matter can be analyzed to diagnose one or more conditions of the patient. According to several examples, endoscope 100 can be suitable for the removal of cancerous or pre-cancerous matter (e.g., carcinoma, sarcoma, myeloma, leukemia, lymphoma and the like), endometriosis evaluation, biliary ductal biopsies, and the like.

However, as mentioned above, the size of the additional device is typically small due to the progressively smaller sizes of endoscope 100, auxiliary scope 134 and the additional device. In examples, lumen 132 of endoscope 100 can typically be on the order of approximately 4.0 mm in diameter, while lumen 136 of auxiliary scope 134 can typically be on the order of approximately 1.2 mm. As such, with conventional devices including baby biopsy forceps, it can be difficult to obtain sufficiently large tissue sample sized to ensure accurate diagnoses without having to repeatedly remove and reinsert the additional device. Likewise, it can be difficult to view the desired matter, e.g., the target tissue, due to multiple reasons including the presence of the tissue retrieval device in the line of sight of the auxiliary scope camera. This thereby makes collection of non-desirable, e.g., non-cancerous, material a possibility. However, with the systems and devices of the present disclosure it is possible to obtain sufficiently large tissue sample sizes with only a single insertion and removal of the additional device, when configured as a tissue retrieval device, biopsy instrument or ductal sampling device of the present disclosure, for example. For example, the tissue retrieval device can be ductal sampling device 200 including textured scraping body 210, as described with reference to FIGS. 6-9 or the other ductal sampling devices described herein.

### Structure of Ductal Sampling Device

Ductal sampling devices of the present application can include an outer sheath having an actuator, such as a knob, at the proximal end. The knob can have a griping shape as shown in the attached drawings or other shapes. The actuator can be attached to the sheath to push and pull the sheath. The actuator can comprise a knob and does not need to include controls or buttons as can be used with clipping devices or forceps.

The ductal sampling devices can include an inner catheter which can be exposed from the outer sheath. The inner catheter can have a textured sampling portion that is designed for agitating the region of interest. This texture can have sharp blades to scrape the tissue better. The texture can also have pockets between the sharp blades for capturing the tissue. The texture can help to improve the ability to agitate tissue by increasing the ability to scratch and trap tissue. The inner catheter can also have an actuator, such as a handle, to push or pull the inner catheter out from the outer sheath, so that the textured sampling portion can be exposed from the outer sheath.

In examples, the outer diameter of the ductal sampling devices can be approximately 1.2 mm or less, so that the device can pass through the working channel of a cholangioscope.

The ductal sampling devices can be made of flexible material (e.g., Nitinol) to have enough flexibility to operate through the endoscope. At least the textured sampling portion can have more stiffness than other parts to have enough strength to scratch the tissue.

FIG. 6 is a side view of a ductal sampling device 200 of the present disclosure comprising inner catheter 202 and outer sheath 204. Inner catheter 202 can comprise handpiece 206, shaft 208, textured scraping body 210 and atraumatic tip 212. Outer sheath 204 can comprise actuator 214 and flexible tube 216. FIG. 7 is a close-up view of outer sheath 204 positioned around shaft 208. FIG. 8 is a close-up view of a distal end of textured scraping body 210 comprising atraumatic tip 212. FIG. 9 is a close-up view of textured scraping body 210 of FIG. 6 comprising teeth 218 and pockets 220. FIGS. 6-9 are discussed concurrently.

Textured scraping body 210 can be configured to do one or both of separate and retrieve biological matter from within a patient after being positioned within the patient by shaft 208. Textured scraping body 210 can be configured to engage target tissue, separate the target tissue from the patient and store separated target tissue for removal from the patient, such as by removal of shaft 208 from the patient. Outer sheath 204 can be positioned around textured scraping body 210 to protect tissue and endoscope components when textured scraping body 210 is not being used and to retain separated target tissue within textured scraping body 210.

Handpiece 206 can comprise any device suitable for facilitating manipulation and operation of ductal sampling device 200. Handpiece 206 can be located at the proximal end of shaft 208 or another suitable location along shaft 208. In examples, handpiece 206 can comprise a pistol grip, a knob, a handlebar grip and the like. Actuator 214 can be attached to outer sheath 204 to expose or cover textured scraping body 210. Actuator 214 can comprise a knob, ring or the like.

Shaft 208 can extend from handpiece 206 and can comprise an elongate member configured to allow textured scraping body 210 to be inserted into a patient. In examples, shaft 208 can be sized for placement within an auxiliary scope, such as auxiliary scope 134 of FIG. 5. In examples, shaft 208 can have a diameter in the range of approximately 2.0 mm to approximately 0.5 mm. In examples, shaft 208 can have a diameter of approximately 1.2 mm. As such, shaft 208 can be inserted into an incision or opening in the epidermis of a patient, through a body cavity of the patient and into an organ. Thus, it is desirable for the diameter or cross-sectional shape of shaft 208, as well as components attached thereto, to be as small as possible to facilitate minimally invasive surgical procedures. Textured scraping body 210 can thus be incorporated into shaft 208 to minimize the size impact on ductal sampling device 200. Shaft 208 can be axially rigid, but resiliently bendable, and formed from a metal or plastic material. In examples, shaft 208 can be fabricated from a nickel titanium alloy, such as nitinol. In examples, shaft 208 can be fabricated from stainless steel, which provides good radiopacity for use with imaging. In examples, shaft 208 can be hollow to receive collected tissue from the textured scraping body 210.

Textured scraping body 210 can be located at or near the distal end of shaft 208 or another suitable location along shaft 208. Atraumatic tip 212 can be positioned at the end of shaft 208 or the end of textured scraping body 210 to provide a blunt tip that prevents puncture through tissue. In examples, atraumatic tip 212 can comprise a cap fabricated of a soft material, such as silicon rubber, highly plasticized PVC or the like. In examples, atraumatic tip 212 can be elongated distally from shaft 208 to allow atraumatic tip 212 to be readily deflected or bent. As such, atraumatic tip 212 can provide visual feedback to a user observing atraumatic tip 212, such as with endoscope 14 (FIG. 2) or auxiliary scope 134 (FIG. 5), for when ductal sampling device 200 has been advanced into an anatomic barrier, e.g., a duct wall. Textured scraping body 210 can be sized to fit within lumen 136 (FIG. 5), for example. In examples, textured scraping body 210 can have the same diameter as shaft 208. In examples, textured scraping body 210 can extend along a length in the range of approximately 5.0 cm to approximately 0.5 cm. In examples, textured scraping body 210 can have a length of approximately 2.0 cm. For example, the length of textured scraping body 210 can be sized to extend across typical sized lesions from with biological matter is to be collected. Furthermore, an excessively long length of textured scraping body 210 can be undesirable because healthy tissue could be unintentionally scraped or abraded. Textured scraping body 210 can be long enough to include multiple rows of teeth 218 and pockets 220. Textured scraping body 210 can comprise a component or device for interacting with a patient, such as those configured to scrape, abrade, grate, cut, slice, pull, saw, punch, twist or auger tissue, and the like. In examples, textured scraping body 210 can comprise a device suitable for removing tissue from a patient. In specific examples, textured scraping body 210 can comprise a device having edges configured to slice tissue from the patient, such as a scraping or grating device. For example, teeth 218 can include scraping edges 219 that overhang pockets 220. Scraping edges 219 of teeth 218 can be sharpened, such as to a point, to facilitate cutting through tissue. Textured scraping body 210 can be configured to physically separate portions of tissue of a patient from other larger portions of tissue in the patient. In additional examples, textured scraping body 210 can be configured to simply collect biological matter from the patient that does not need physical separation, such as mucus or fluid, or biological matter that has already been separated, such as by another device. In examples, textured scraping body 210 can be configured to physically separate portion of tissue of a patient for retrieval with ductal sampling device 200. In the illustrated example, textured scraping body 210 can comprise a cylindrical body with annular rings of material forming teeth 218, which additionally form annular shaped voids or spaces recessed into shaft 208 to comprise pockets 220. Teeth 218 and pockets 220 can circumscribe a central axis of shaft 208. As such, teeth 218 can perform scraping along a three-hundred-sixty-degree arc relative to the central axis of shaft 208. A plurality of rows of teeth 218 and pockets 220 can be included to provide a length of scraping capability. As such, textured scraping body 210 can be configured to have features that allow for direct storing of collected biological matter or tissue.

Handpiece 206 can be operated by a user to operate textured scraping body 210. Handpiece 206 can be used to manipulate shaft 208 to push textured scraping body 210 against target tissue. For example, shaft 208 can be rotated, oscillated, reciprocated and the like move textured scraping body 210 along the target tissue to cause textured scraping body 210 to separate sample tissue from the target tissue attached to the patient. Actuator 214 can be coupled to handpiece 206 and can be configured to operate outer sheath 204. Actuator 214 can comprise any type of device suitable for axially translating outer sheath 204. In examples, actuator 214 can comprise one or more of a lever, a trigger, a joystick, a button, a wheel and the like, as well as combinations thereof. Flexible tube 216 of outer sheath 204 can surround shaft 208 and textured scraping body 210. Flexible tube 216 can comprise a polymer tube or the like that can have an internal lumen slightly larger than shaft 208 and textured scraping body 210 to allow inner catheter 202 to slide therein. Outer sheath 204 can be used to provide stiffness to shaft 208, such as proximate handpiece 206 to facilitate insertion into anatomy. Outer sheath 204 can be advanced distally along shaft 208 using actuator 214 to position flexible tube 216 over textured scraping body 210 and outer sheath 204 can be advanced proximally along shaft 208 to cover textured scraping body 210.

Textured scraping body 210 can be fully retracted into lumen 136 (FIG. 5). As such, camera lens 137 can be freely moved by manipulation of shaft 208 to position target tissue within field of view of camera lens 137. However, when it is desired to extend textured scraping body 210 from lumen 136, textured scraping body 210 can become positioned within the field of view of camera lens 137.

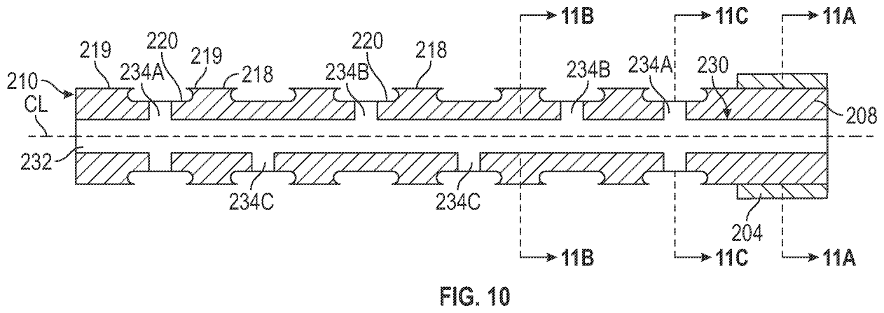

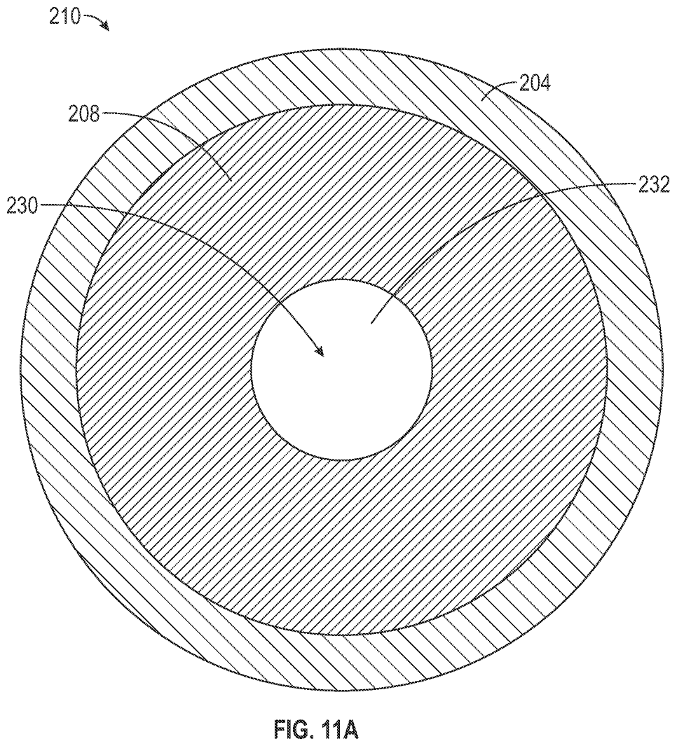

FIG. 10 is a side cross-sectional view of textured scraping body 210 of FIG. 9 showing internal passage 230. Internal passage 230 can comprise longitudinal lumen 232, side ports 234A, side ports 234B and side ports 234C. Internal passage 230 can be configured to receive collected biological matter from pockets 220. Internal passage 230 can be optionally included in inner catheter 202.

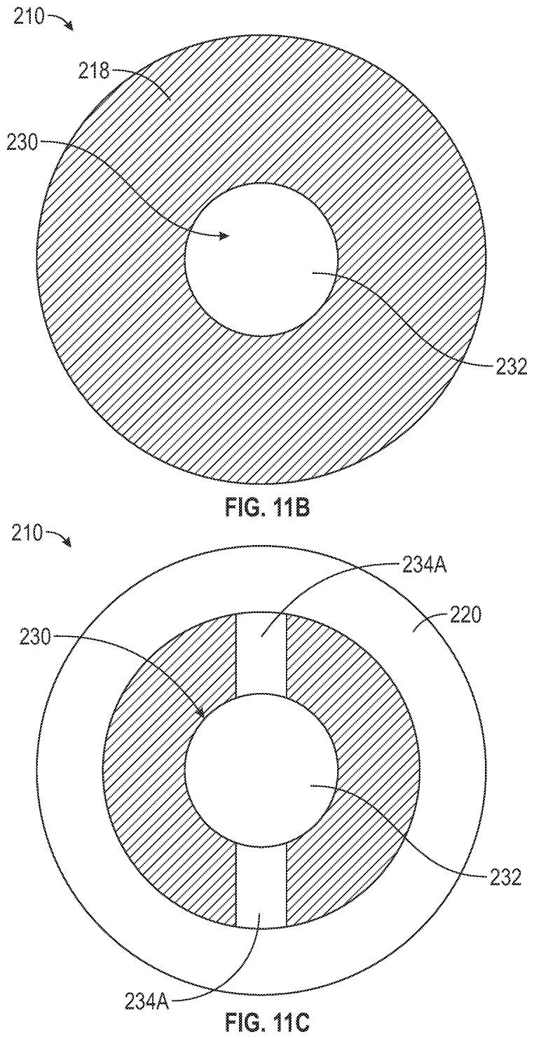

FIG. 11A is a cross-sectional view through textured scraping body 210 of FIG. 10 showing outer sheath 204 covering shaft 208 and internal passage 230 within shaft 208. FIG. 11B is a cross-sectional view through textured scraping body 210 of FIG. 10 showing teeth 218. FIG. 11C is a cross-sectional view through textured scraping body 210 of FIG. 10 showing pockets 220. FIGS. 10-11C are discussed concurrently.

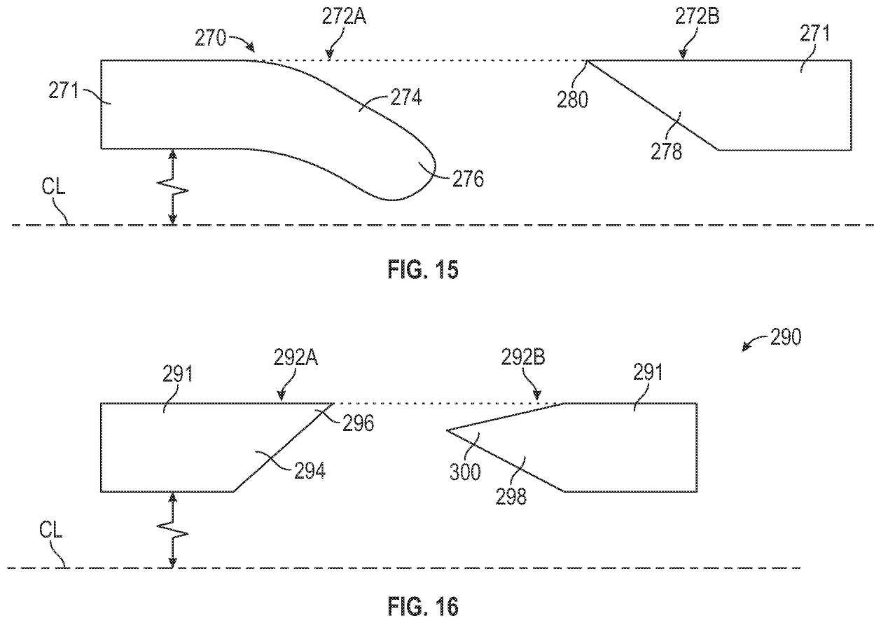

Textured scraping body 210 of FIG. 10 can be configured similarly as textured scraping body 210 of FIG. 6, such as by including teeth 218 and pockets 220. As discussed in greater detail with reference to FIGS. 15 and 16, teeth 218 can include scraping edges 219 that can be configured to slice, cut, abrade, grate or agitate tissue. Some of scraping edges 219 can be configured to slide over or across tissue without causing any irritation.

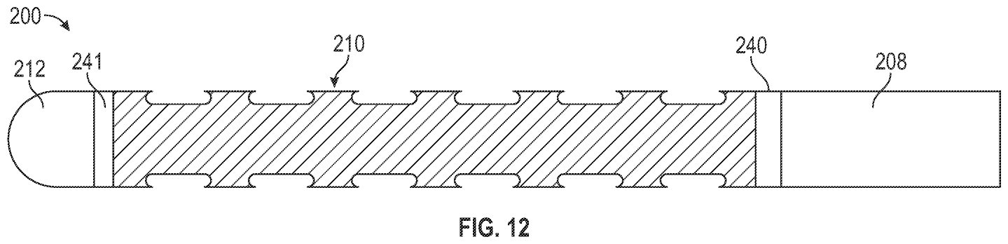

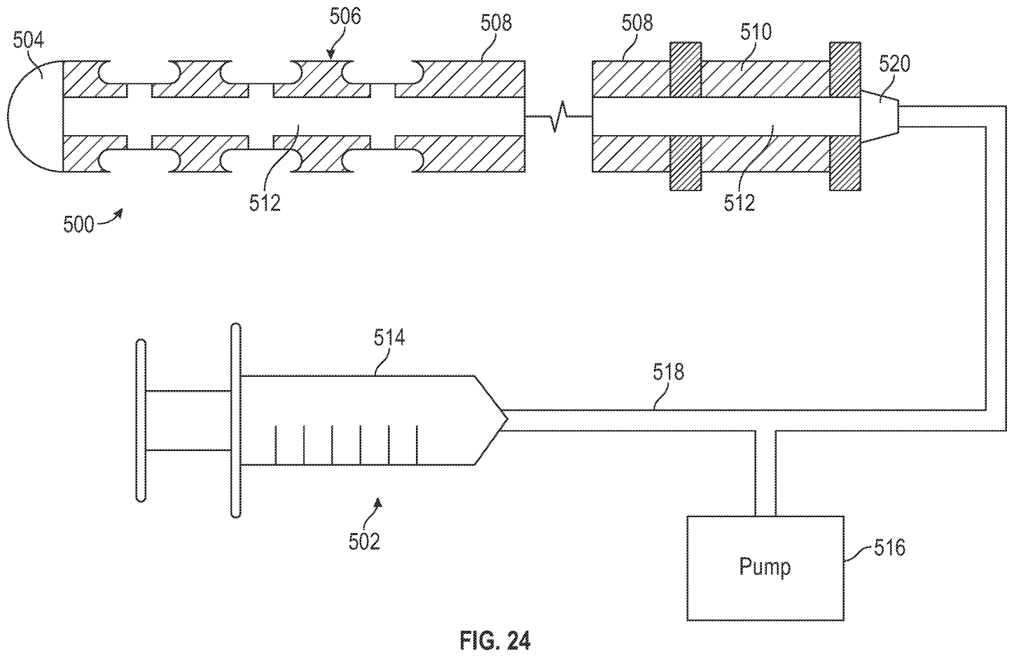

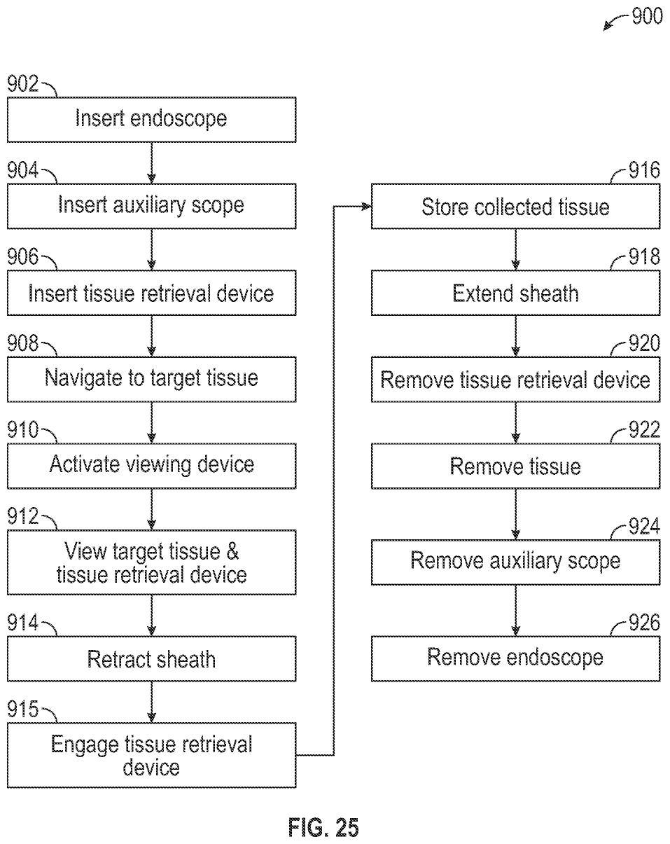

Longitudinal lumen 232 can comprise an axially extending passage that extends through textured scraping body 210. In examples, longitudinal lumen 232 can extend over the entire length of textured scraping body 210 to connect to each of pockets 220 therein. Longitudinal lumen 232 need not extend all the way to atraumatic tip 212. However, longitudinal lumen 232 can extend to atraumatic tip 212 and atraumatic tip 212 can be removable to access internal passage 230. Likewise, longitudinal lumen 232 need not extend into shaft 208, but can do so to facilitate access to internal passage 230, such as be decoupling of shaft 208 from textured scraping body 210 (FIG. 12) or connection to a removal device (FIG. 14) or system (FIG. 24). In examples, longitudinal lumen 232 can extend all the way to handpiece 206 (FIG. 6) and in additional examples can extend through handpiece 206, as can be seen in FIG. 24. Longitudinal lumen 232 can comprise a cylindrical passage extending along centerline CL. The diameter or cross-section of longitudinal lumen 232 can be sufficiently large to allow biological matter to pass freely therethrough without weakening of shaft 208.

Side ports 234A, side ports 234B and side ports 234C can extend radially inward from one or more of pockets 220 toward centerline CL. Side ports 234A through side ports 234C can comprise cylindrical bores extending into pockets 220. However, side ports 234A through side ports 234C can be angled from pockets 220 toward centerline CL in non-radial directions along axes that do not pass through centerline CL. As such, each of pockets 220 can comprise one or more of side ports 234A through side ports 234C to collect biological matter at different radial positions relative to centerline CL. Side ports 234A can extend radially all the way across textured scraping body 210 from one of pockets 220 to another of pockets 220. Side ports 234A can collect biological matter from opposing sides of pockets 220 or opposite sides of annularly shaped pockets such as pockets 220. Side ports 234B and side ports 234C can extend radially inward from one side of pockets 220 to prevent biological matter from passing through textured scraping body 210. Different combinations of one or more of side ports 234A, side ports 234B and side ports 234C can be used.

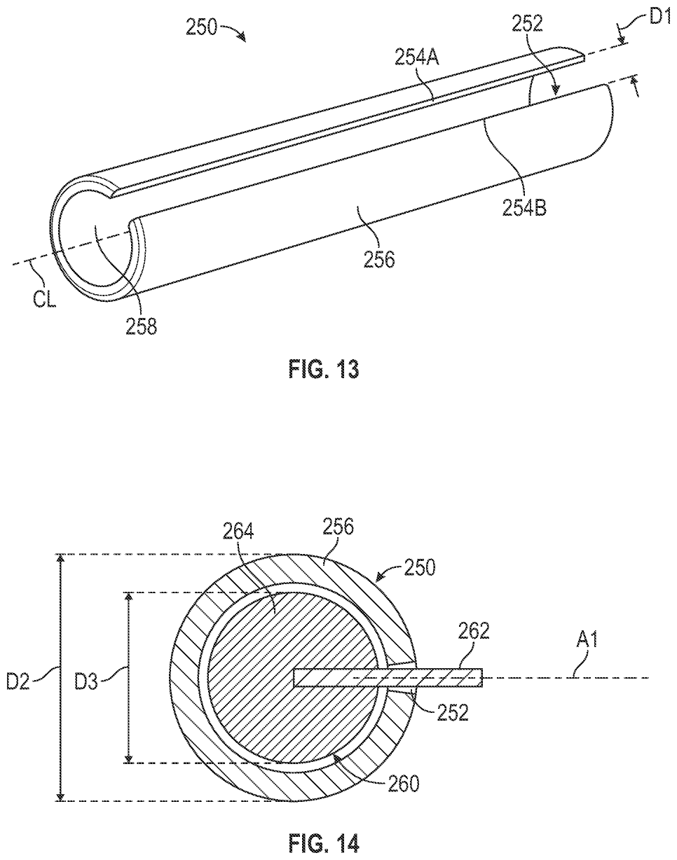

Side ports 234A through side ports 234C can increase the load capacity of textured scraping body 210 by allowing biological matter to be stored radially inward of pockets 220. Side ports 234A through side ports 234C can additionally allow biological matter from pockets 220 to pass into longitudinal lumen 232. Longitudinal lumen 232 can provide additional storage space for textured scraping body 210. As discussed with reference to FIGS. 13 and 14, internal passage 230 can facilitate collection of the biological matter from textured scraping body 210. As discussed with reference to FIG. 24, internal passage 230 can facilitate collection of biological matter using a fluid injection or suction system.

FIG. 12 is a schematic side view of ductal sampling device 200 of FIG. 6 showing textured scraping body 210 connected to shaft 208 via break-away section 240. Break-away section 240 can comprise a segment of material to allow textured scraping body 210 to be detached from shaft 208. In examples, break-away section 240 can comprise perforations within shaft 208. In examples, break-away section 240 can comprise a weakened portion of shaft 208, such as a thinned-down section. In examples, break-away section 240 can comprise a different material than shaft 208 to facilitate tearing or slicing through ductal sampling device 200, such as a plastic, silicone or rubber material. In examples, break-away section 240 can comprise a plurality of frangible struts connecting shaft 208 and textured scraping body 210. In examples, break-away section 240 can comprise a releasable coupling, such as a threaded coupling or a snap fit coupling, between shaft 208 and textured scraping body 210. In examples, atraumatic tip 212 can be connected to textured scraping body 210 via coupler 241 comprising a break-away or releasable coupling similar to break-away section 240.

After textured scraping body 210 is used to collect biological matter such that pockets 220 are filled or partially filled with biological matter, ductal sampling device 200 can be withdrawn from the anatomy, and break-away section 240 can be broken or severed to remove textured scraping body 210 from shaft 208. Thereafter, textured scraping body 210 can be transported to a laboratory where the biological matter can be collected and analyzed. In examples, textured scraping body 210 filled with biological matter can be deposited into a fluid bath to collect the tissue cells for analysis. In examples, atraumatic tip 212 can be removed form textured scraping body 210 before tissue analysis is performed. In examples, passages within textured scraping body 210, such as internal passage 230, can facilitate removal of biological material by allowing fluids used in biopsy procedures to pass through textured scraping body 210.

FIG. 13 is a perspective view of shaft 250 of a ductal sampling device comprising slit 252. Slit 252 can comprise first edge 254A and second edge 254B. Shaft 250 can comprise annular body 256 and lumen 258. Shaft 250 can comprise shaft 208 of FIG. 6 and FIG. 11, for example. In examples, shaft 250 can comprise a rolled sheet of material or a seamless tube that has been cut. In examples, slit 252 can extend along shaft 208 from textured scraping body 210 to handpiece 206 (FIG. 6). In other examples, slit 252 can extend over only a portion of shaft 250. In examples, handpiece 206 can be removable to facilitate access to internal passage 230 (FIG. 10), e.g., lumen 258. Slit 252 can be configured to allow another instrument to interact with internal passage 230. Slit 252 can space first edge 254A and second edge 254B apart from each other distance D1. Distance D1 can be small to inhibit or prevent biological matter from falling out of or otherwise leaving lumen 258. However, distance D1 can be large enough to allow a portion of an instrument to pass therethrough, as shown in FIG. 14. In examples, distance D1 can be approximately 1.0 mm. In examples, annular body 256 can be configured to spring shut to close distance D1 so that first edge 254A contacts second edge 254B. However, in such examples, an instrument can be inserted between first edge 254A and second edge 254B to open slit 252.

FIG. 14 is a cross-sectional view of shaft 250 of FIG. 13 having scraper 260 extending into slit 252. Scraper 260 can comprise handle 262 and paddle 264. Lumen 258 of shaft 250 can have diameter D2 and paddle 264 can have diameter D3. Diameter D3 of paddle 264 can be slightly smaller than diameter D2 of lumen 258 to allow paddle 264 to move within lumen 258. In examples, paddle 264 can contact walls of annular body 256 forming lumen 258 to form a scal therebetween. In examples, paddle 264 can be spaced from walls of annular body 256 forming lumen 258. Paddle 264 can comprise a disk or plate that can be advanced through lumen 258 to push biological matter through shaft 250. Paddle 264 can have sufficient rigidity to push biological matter along lumen 258. Paddle 264 and handle 262 can be thin, such as only being approximately 0.5 cm to approximately 1.0 cm thick.

Handle 262 can extend from paddle 264 through slit 252 along axis A1. Handle 262 can comprise an elongate body such as a shaft or tube to allow for grasping by an operator of a ducal sampling device. Handle 262 can have sufficient rigidity to push biological matter along lumen 258. Handle 262 can have a thickness slightly smaller than distance D1. In examples, handle 262 can contact first edge 254A and second edge 254B to form a seal therebetween. In examples, handle 262 can be spaced from first edge 254A and second edge 254B.

After textured scraping body 210 is used to collect biological matter such that pockets 220 are filled or partially filled with biological matter, ductal sampling device 200 can be withdrawn from the anatomy, and scraper 260 can be inserted into lumen 258. In examples, an atraumatic tip, such as atraumatic tip 212, can be separated from annular body 256 to allow paddle 264 to be positioned within lumen 258. Thus, paddle 264 can be positioned with lumen 258 with the faces of paddle 264 perpendicular to centerline CL and advanced therethrough. In examples, paddle 264 can be aligned with slit 252, such as by positioning the faces of paddle 264 parallel to centerline CL, to pass paddle 264 into lumen 258 and can then be rotated about axis A1 to be transverse to the central axis of lumen 258. Thereafter, paddle 264 can be advanced proximally using handle 262 to push biological matter toward handpiece 206 (FIG. 6) or distally to push biological matter toward atraumatic tip 212. In examples, handpiece 206 and atraumatic tip 212 can be removed to allow the biological matter to be pushed out of lumen 258. In examples, a proximal portion of shaft 250 can include an access port or window to allow the biological matter to be extracted using suction or another instrument, such as a spoon, scoop or spatula, such that slit 252 need not extend all the way to handpiece 206 and handpiece 206 need not be removable. The biological material can be transferred to a container and transported to a laboratory where the biological matter can be analyzed.

FIG. 15 is a schematic side view of textured scraping body 270 comprising shaft 271 having first edge 272A and second edge 272B of the present disclosure. In the illustrated example, first edge 272A can comprise a sliding edge and second edge 272B can comprise a cutting edge opposing the sliding edge. First edge 272A can comprise body 274 that is deflected inward toward centerline CL and that terminates in blunt end 276. Second edge 272B can comprise body 278 that extends straight axially outward from shaft 271 and that terminates in point 280. Shaft 271 can comprise an annular body revolved around centerline CL. As such, FIG. 15 illustrates only half of shaft 271. The distance of shaft 271 from centerline CL is not necessarily drawn to scale in FIG. 15.

Textured scraping body 270 of FIG. 15 can be configured for unidirectional scraping. That is, textured scraping body 270 can be configured to scrape biological matter when advanced in one of the proximal and distal directions and to not scrape when advanced in the other direction. As such, only one of first edge 272A and second edge 272B is configured for scraping.

In the example of FIG. 15, first edge 272A is configured to not perform scraping. First edge 272A is blunted and angled inward toward centerline CL to prevent contacting biological matter. Specifically, blunt end 276 can be rounded or otherwise include curved surfaces and body 274 can be angled inward so that first edge 272A only presents curved surfaces to the anatomy when being pushed to the right in FIG. 15. As such, first edge 272A can slide across or over tissue without agitating or irritating tissue. In examples, first edge 272A can include only one of a blunt tip and an angled body. For example, first edge 272A can include blunt end 276 without being curved inward by body 274, or first edge 272A can include body 274 that is curved inward such that the tip or end of body 274 does not contact tissue and thus can have an edge that does not engage tissue (similar to second edge 292B of FIG. 16).

In the example of FIG. 15, second edge 272B is configured to perform scraping. Second edge 272B is sharp and projects axially to contact biological matter. Specifically, point 280 can be sharpened or otherwise include an edged surface and body 278 can be angled so that second edge 272B presents a scraping surface to the anatomy when being pushed to the left in FIG. 15. Point 280 can axially align with the outer surface of shaft 271 so as to not project radially outward. As such, point 280 will not interfere with outer sheath 204 (FIG. 6), auxiliary scope 134 (FIG. 5) or endoscope 14 (FIG. 1). Point 280 can comprise an edge suitable for separating biological matter from base tissue. In example, point 280 can comprise a sharpened edge, such as a razor or a blade. However, point 280 need not comprise a sharpened edge and can comprise a rough edge that can agitate or abrade tissue.

In examples, where first edge 272A is distal of second edge 272B (e.g., where handpiece 206 [FIG. 6] is to the right in FIG. 15), textured scraping body 270 can be configured to scrape biological matter when pushed distally and not scrape biological matter when pulled proximally.

In examples, where second edge 272B is distal of first edge 272A (e.g., where handpiece 206 [FIG. 6] is to the left in FIG. 15), textured scraping body 270 can be configured to scrape biological matter when pulled proximally and not scrape biological matter when pushed distally.

FIG. 16 is a schematic side view of textured scraping body 290 comprising shaft 291 having first edge 292A and second edge 292B of the present disclosure. In the illustrated example, first edge 292A can comprise a cutting edge and second edge 272B can comprise a sliding edge. First edge 292A can comprise body 294 that extends straight axially outward from shaft 291 and that terminates in point 296. Second edge 292B can comprise body 298 that is deflected inward toward centerline CL and that terminates in sliding end 300. Shaft 291 can comprise an annular body revolved around centerline CL. As such, FIG. 16 illustrates only half of shaft 291. The distance of shaft 291 from centerline CL is not necessarily drawn to scale in FIG. 16.

Textured scraping body 290 of FIG. 16 can be configured for unidirectional scraping. That is, textured scraping body 290 can be configured to scrape biological matter when advanced in one of the proximal and distal directions and to not scrape when advanced in the other direction. As such, only one of first edge 292A and second edge 292B is configured for scraping.

In the example of FIG. 16, first edge 292A is configured to perform scraping. First edge 292A is sharp and projects axially to contact biological matter. Specifically, point 296 can be sharpened or otherwise include an edged surface and body 294 can be angled so that first edge 292A presents a scraping surface to the anatomy when being pushed to the right in FIG. 16. Point 296 can axially align with the outer surface of shaft 271 so as to not project radially outward. As such, point 296 will not interfere with outer sheath 204 (FIG. 6), auxiliary scope 134 (FIG. 5) or endoscope 14 (FIG. 1). Point 296 can comprise an edge suitable for separating biological matter from base tissue. In example, point 296 can comprise a sharpened edge, such as a razor or a blade. However, point 296 need not comprise a sharpened edge and can comprise a rough edge that can agitate or abrade tissue.

In the example of FIG. 16, second edge 292B is configured to not perform scraping. Second edge 292B can be angled inward toward centerline CL to prevent contacting biological matter. Specifically, sliding end 300 can include an edge that projects inward so that second edge 292B only presents inclined or sloped surfaces to the anatomy when being pushed to the left in FIG. 16. As such, second edge 292B can slide across or over tissue without agitating or irritating tissue. In examples, second edge 292B can include a blunted tip. For example, sliding end 300 can additionally include a rounded tip.

In examples, where first edge 292A is distal of second edge 292B (e.g., where handpiece 206 [FIG. 6] is to the right in FIG. 16), textured scraping body 290 can be configured to scrape biological matter when pulled proximally and not scrape biological matter when pushed distally.

In examples, where second edge 292B is distal of first edge 292A (e.g., where handpiece 206 [FIG. 6] is to the left in FIG. 16), textured scraping body 290 can be configured to scrape biological matter when pushed distally and not scrape biological matter when pulled proximally.

FIG. 15 and FIG. 16 illustrate examples of edges that can be used with teeth 218 of FIG. 6 or scraping edges 219 of FIG. 10. The various edges shown in FIG. 15 and FIG. 16, i.e., first edge 272A, second edge 272B, first edge 292A and second edge 292B can be used in different combinations. In examples, textured scraping body 210, for example, can be configured to perform unidirectional scraping or bidirectional scraping. Thus, in examples, at least one edge of opposing edges, i.e., first edge 272A and second edge 272B or first edge 292A and second edge 292B, can be configured to scrape or both edges of opposing edges, i.e., first edge 272A and second edge 272B or first edge 292A and second edge 292B, can be configured to scrape.

However, in some examples, neither of the edges of opposing edges can be configured to scrape. In examples, textured scraping body 210 can be configured so that not every tooth of teeth 218 scrapes. For example, every other tooth of teeth 218 of FIG. 10 can be configured to scrape or textured scraping body 210 can be configured to scrape and collect previously cut or loosened biological material.

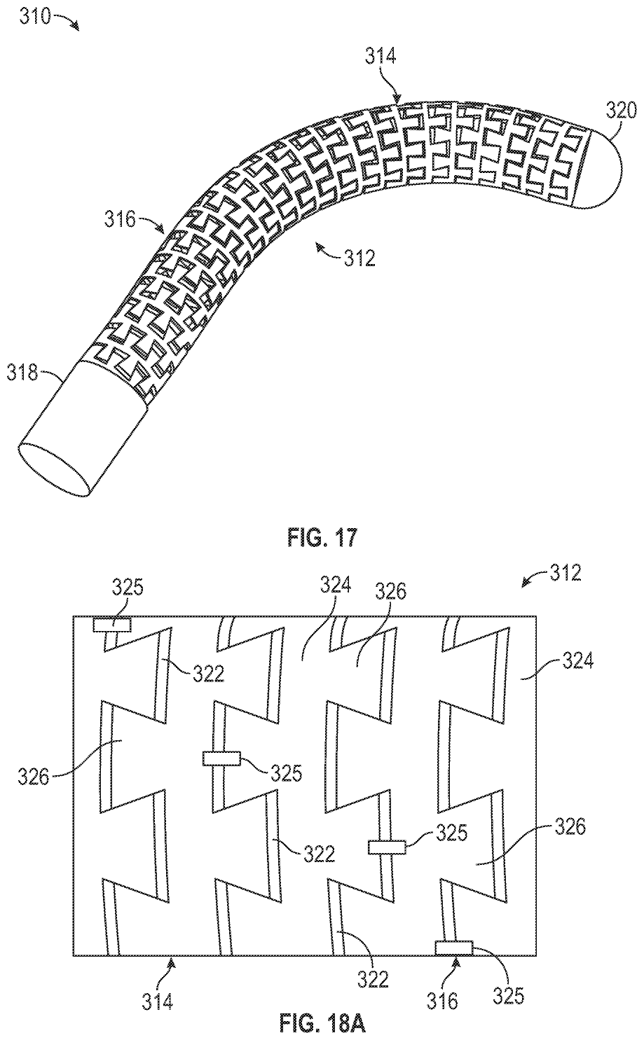

FIG. 17 is a perspective view of ductal sampling device 310 of the present disclosure comprising flexible scraping section 312 including flexible linkages or flexible links 314 spaced by cut-outs or trenches 316. Flexible scraping section 312 can be positioned between shaft 318 and tip 320.

Flexible scraping section 312 can be configured to allow the textured scraping bodies of the present disclosure to bend to facilitate insertion through anatomy and to facilitate collection of biological matter. Flexible scraping section 312 can comprise an extension of the material of shaft 318 or can comprise a separate component attached to shaft 318. Tip 320 can be attached to flexible scraping section 312 and can be configured as an atraumatic tip, such as is described herein. Flexible links 314 can simultaneously provide bending and cutting or scraping functionality to ductal sampling device 310. For example, trenches 316 can provide space within the material of flexible scraping section 312 to allow for bending, while edges of flexible links can be sharpened to separate biological matter.

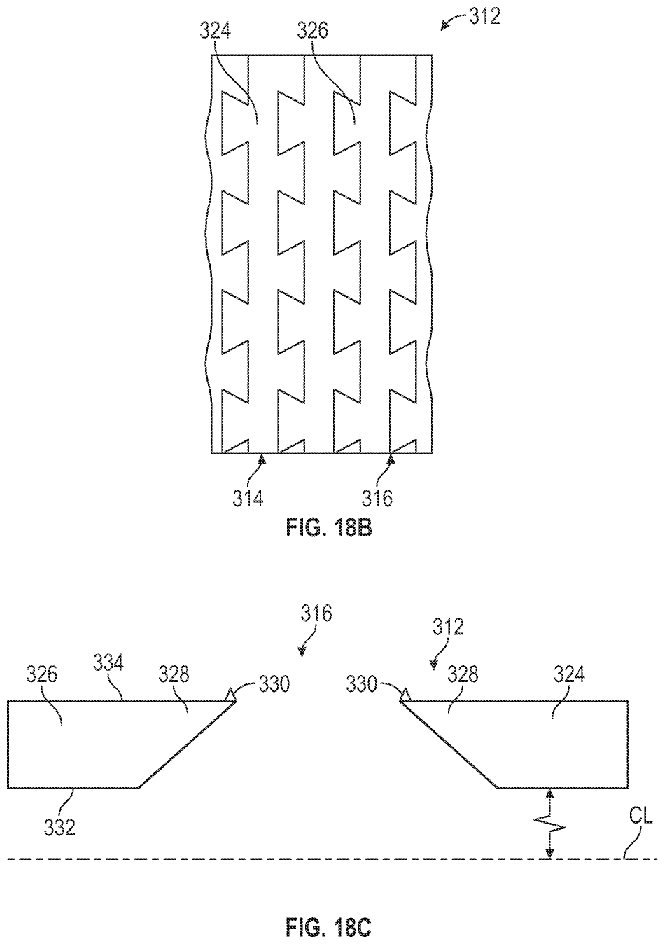

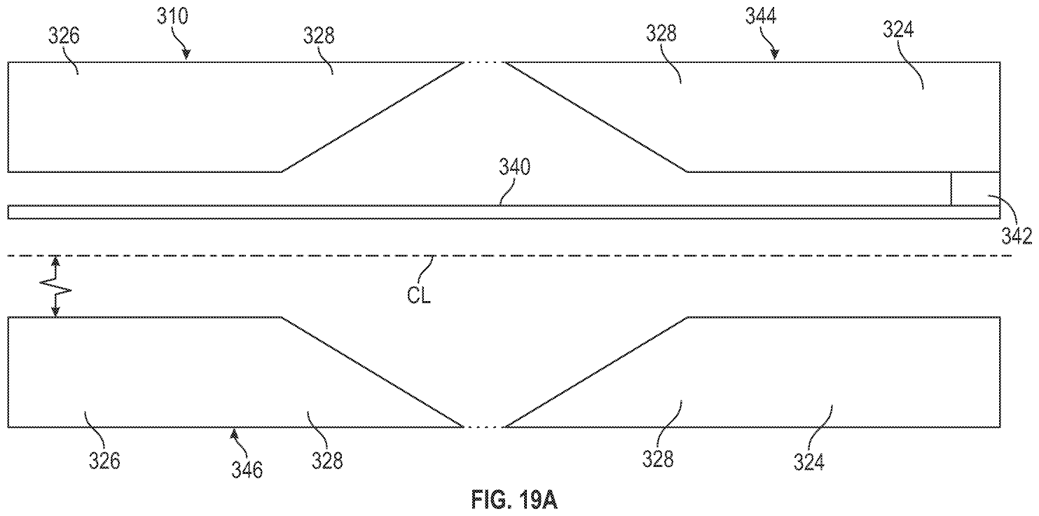

Flexible links 314 can comprise interlocking bodies that are spaced apart to allow for radially bending of flexible scraping section 312 relative to the central axis of shaft 318 (e.g., centerline CL in FIG. 18C), but that are connected axially to provide rigidity to flexible scraping section 312. Upon bending, the interconnected bodies will eventually contact each other to prevent further bending.

FIG. 18A is a top view of flexible links 314 of FIG. 17 in an expanded state at rest such that trenches 316 form gaps 322. FIG. 18B is a top view of flexible links 314 of FIG. 18A in a contracted state under compression. Flexible links 314 can comprise circumferential strips 324 and lobes 326. FIGS. 18A and 18B are discussed concurrently.

Circumferential strips 324 can comprise annular bodies that provide circumferential stiffness to ductal sampling device 310. The annular bodies can be axially connected to each other at different circumferential positions along the length of flexible scraping section 312 using extensions 325 to provide axial stiffness without interfering with the ability to bend. Lobes 326 can comprise trapezoidal shaped bodies having an end surface that extends circumferentially and two side surfaces that are oblique to the central axis of shaft 318. Circumferential strips 324 can include lobes 326 that alternately extend from opposite axial sides. Lobes 326 can fit into oppositely shaped cut-outs provided by trenches 316 on an adjoining circumferential strip 324. Thus, as shaft 318 is flexed, the trapezoidal bodies can slide against adjoining trapezoidal bodies of an opposite circumferential strip 324 until contact is made. Extensions 325 can be provided in at least one circumferential location between adjacent circumferential strips 324 to maintain continuity of shaft 318.

FIG. 18C is a schematic side view of flexible links 314 of FIG. 17 incorporating scraping edges 328. Scraping edges 328 can comprise burrs 330. Scraping edges 328 can be configured as any of the scraping edges of FIGS. 15 and 16.

Flexible scraping section 312 can be fabricated from a flat metal sheet that is cut to the pattern of FIGS. 18A and 18B. In examples, trenches 316 can be formed by laser cutting, such as by applying the laser beam to first side 332. Furthermore, laser cutting can cause burrs 330 to form at scraping edges 328 when penetrating through the material. Burrs 330 can additionally be used to provide scraping functionality.

In additional examples, trenches 316 can be formed by etching. For example, an acid can be deposited on first side 332 of shaft 318 to form trenches 316. The acid can corrode or otherwise eat-away the material of shaft 318 where contact is made. Gravity or another force can cause the acid to move through the material toward second side 334. As the acid moves through the material, the acid can be consumed causing a narrowing of the etching path, resulting in scraping edges 328 being formed as the acid penetrates through second side 334.

In additional examples, after laser cutting or acid etching, shaft 318 can be subject to a rolling process to push one or more of scraping edges 328 inward, similar to what is discussed with reference to FIGS. 15 and 16, to produce a non-scraping edge to produce uni-directional scraping, for example.

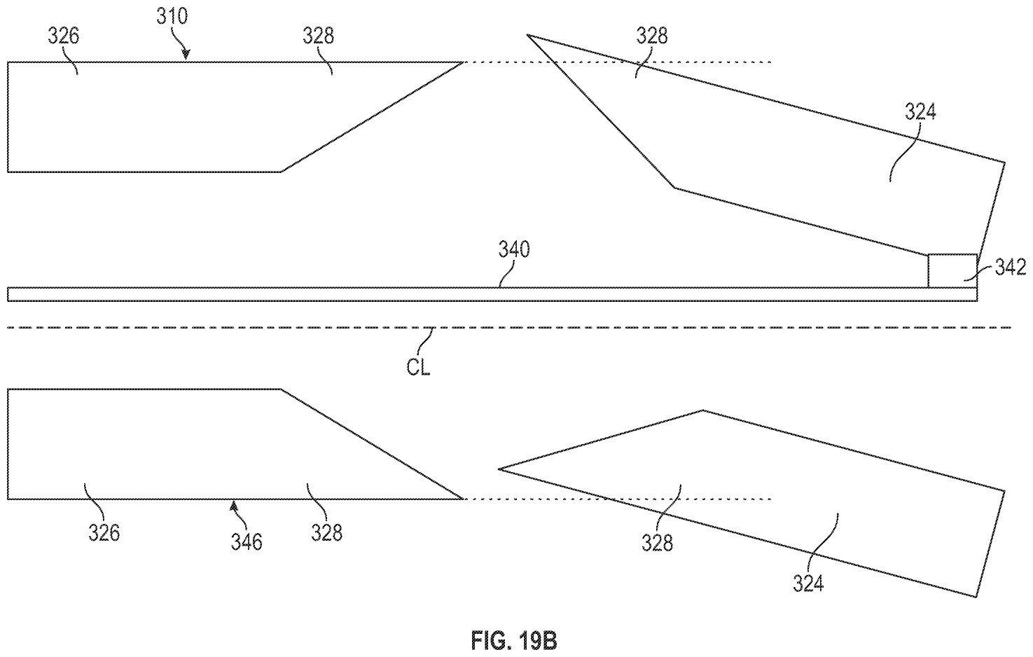

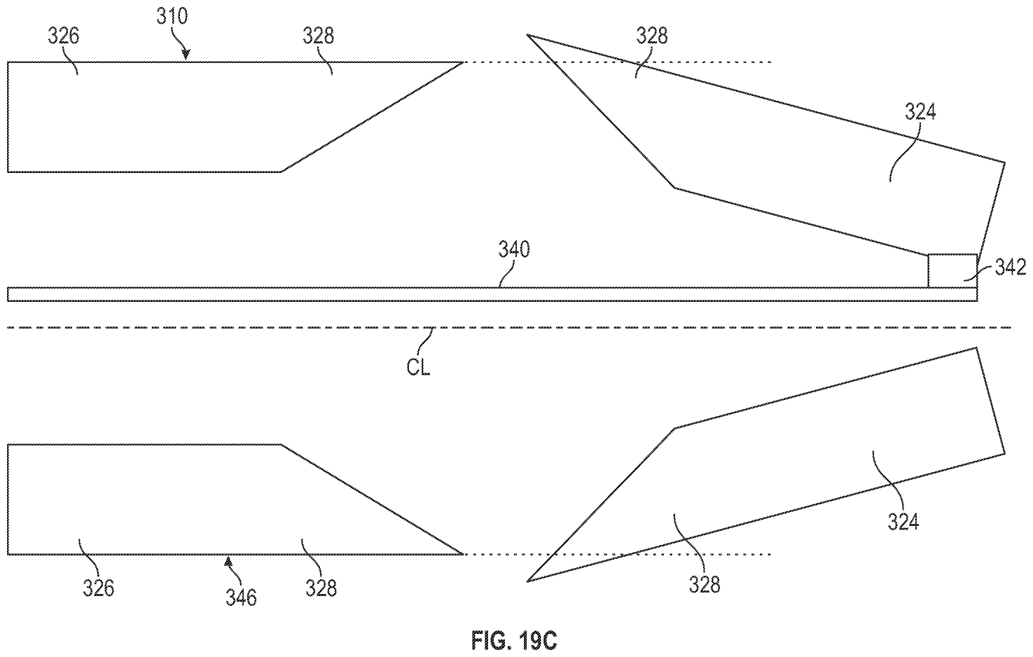

FIG. 19A is a schematic side view of a flexible scraping section 312 for ductal sampling device 310 of the present disclosure including activation wire 340. Flexible scraping section 312 can comprise scraping edges 328 extending from opposing portions of lobe 326 and circumferential strip 324. Activation wire 340 can be attached to a distal portion of flexible scraping section 312 by connector 342. Activation wire 340 is described with reference to ductal sampling device 310 and flexible scraping section 312 of FIGS. 17-18C, but can be used in conjunction with other ductal sampling devices and textured scraping bodies of the present disclosure. Only one activation wire 340 is illustrated, but multiple activation wires can be used if desired.

FIG. 19A shows opposing sides of ductal sampling device 310 comprising upper side 344 and lower side 346, as labeled with reference to the orientation of FIG. 19A. Ductal scraping device 310 can be connected to an activator to induce shaping, bending, bowing or bulging of ductal sampling device 310. In examples, the activator can comprise a pull wire or activation wire 340. In examples, the activator can comprise sheath 204 (FIG. 6). For example, sheath 204 can be connected to a distal portion of flexible scraping section 312 by a length of wire. Sheath 204 can be configured to advance proximally so that the distal tip of sheath 204 is moved partially of fully past flexible scraping section 312 before the wire becomes tensioned. Once the wire is tensioned further proximal movement of sheath 204 can pull on the distal portion of flexible scraping section 312, as discussed below. Thus, in examples, rearward or proximal movement of activation wire 340 or sheath 204 can cause rearward or proximal movement of the distal portion of flexible scraping section 312, thereby expose one or more or scraping edges 328, as discussed below. Once tension in activation wire 340 or sheath 204 is released, flexible scraping section 312 can return to the default straight configuration.

FIG. 19B is a schematic side view of flexible scraping section 312 of FIG. 19A with activation wire 340 pulled to induce flexing of ductal sampling device 310. FIG. 19B shows flexible scraping section 312 in a curved or bended state where one of scraping edges 328 on upper side 344 is exposed outward beyond the perimeter of shaft 318, while one of scraping edges 328 on lower side 346 is withdrawn inward of the perimeter of shaft 318. Thus, with reference to the orientation of FIG. 19B, the upper portion of flexible scraping section 312 can be engaged with tissue to cause the upper scraping edge 328 exposed beyond the perimeter of shaft 318 to scrape against anatomy and separate tissue specimens.

FIG. 19C is a schematic side view of flexible scraping section 312 of FIG. 19A with activation wire 340 pulled to induce bulging of ductal sampling device 310. FIG. 19C shows flexible scraping section 312 in a bowed, bulged or lanterned state where one of scraping edges 328 on upper side 344 is exposed outward beyond the perimeter of shaft 318 and one of scraping edges 328 on lower side 346 is exposed outward beyond the perimeter of shaft 318. Thus, with reference to the orientation of FIG. 19C, the upper and lower portions of flexible scraping section 312 can be engaged with tissue to cause the upper and lower scraping edges 328 exposed beyond the perimeter of shaft 318 to scrape against anatomy and separate tissue specimens.

With reference to FIG. 17, shaft 318 can additionally include pre-curvature to facilitate placement of ductal sampling device 310 and to facilitate engagement of flexible scraping section 312 with tissue. In examples, flexible scraping section 312 can have a pre-curvature in an S-shape, a J-shape or a question-mark-shape. As such, the shaping of flexible scraping section 312 described with reference to FIGS. 19B and 19C can be achieved without an activation source. In examples, with reference to FIG. 6, outer sheath 204 can be used to straighten flexible scraping section 312 during insertion and retraction of ductal sampling device 312. However, when flexible scraping section 312 is at the site of target tissue, outer sheath 204 can be retracted to allow flexible scraping section 312 and shaft 318 to move into the shape of the pre-curvature. As discussed herein, the pre-curvature can facilitate exposure or extension of scraping edges 328 to better engage with tissue.

In order to introduce pre-curvature into flexible scraping section 312, the material of flexible scraping section 312 can be positioned around a shape mandrel that can be shaped to the desired curvature and then heated to allow the material to take the shape of the mandrel by allowing the polymer crystals to form at a slow growth rate to retain the curvature of the mandrel. For example, flexible scraping section 312 can be heated at about 280º Fahrenheit (˜138º Celsius). In examples, manufacturing of guide catheter 100 and the pre-curvatures imparted therein can be performed using conventional manufacturing techniques known in the art.

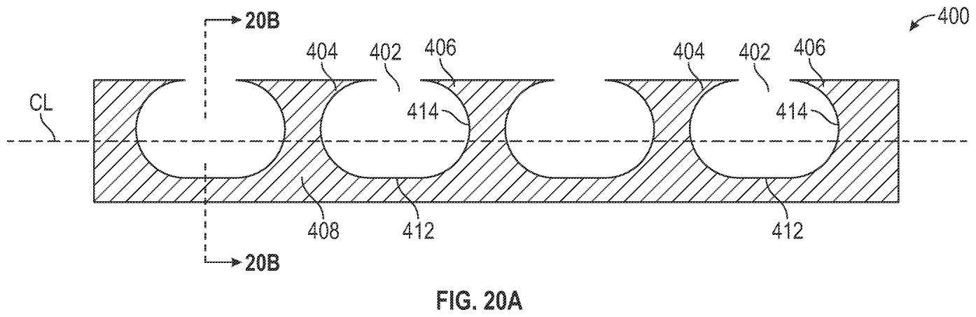

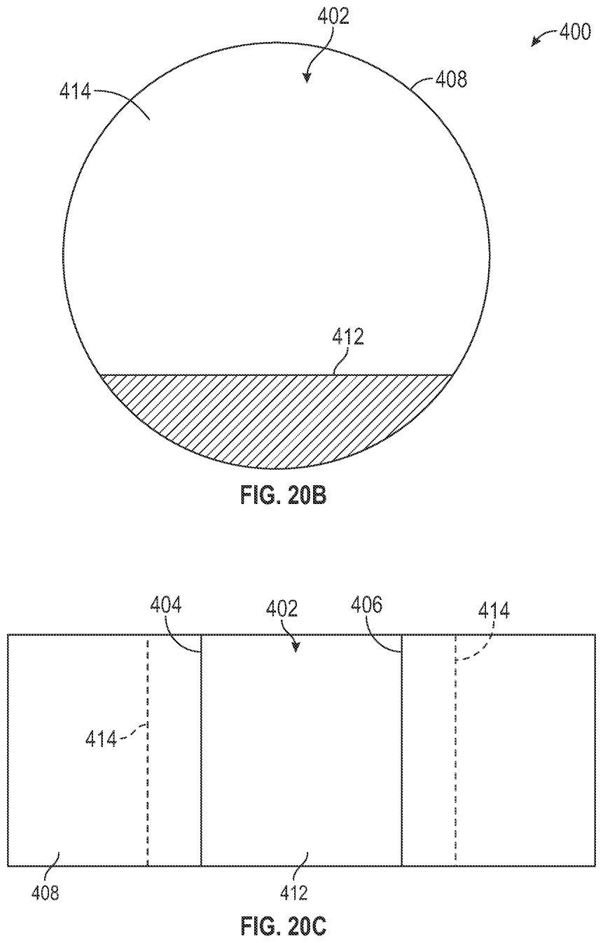

FIG. 20A is a schematic side view of textured scraping body 400 for a ductal sampling device of the present disclosure comprising enlarged pockets 402 forming scraping edge 404 and scraping edge 406 that are aligned along shaft 408 to face in a common direction. FIG. 20B is a cross-sectional view through one of the enlarged pockets 402 of FIG. 20A showing trough 412 and sidewall 414 of the illustrated trough. FIG. 20C is a top view of one of the enlarged pockets 402 of FIG. 20A showing scraping edge 404 and scraping edge 406 overhanging the illustrated trough. FIGS. 20A-20C are discussed concurrently.

Enlarged pockets 402 can be formed within shaft 408 to form scraping edges 404 and 406. In cross-section (e.g., FIG. 20A), enlarged pockets 402 can be similar in shape and function as scraping edges 219 of FIG. 10. However, rather than comprising annular bodies recessed into shaft 408 to surround centerline CL, enlarged pockets 402 can extend straight through shaft 408 to form an axial passage having an inlet and outlet. Enlarged pockets 402 can comprise troughs 412 and sidewalls 414. Troughs 412 can be located on the opposite side of centerline CL than scraping edges 404 and 406. However, pockets 220 of FIG. 10 are located completely on one side of the centerline. As such, enlarged pockets 402 can be larger in volume than pockets 220 of FIG. 10.

Additionally, scraping edge 404 and scraping edge 406 can allow textured scraping body 400 to perform tissue scraping in a selectable radial direction. Scraping edge 404 and scraping edge 406 can perform scraping along less than a three-hundred-sixty-degree arc to have a radial orientation relative to centerline CL. The textured scraping body 400 can allow a surgeon to only agitate tissue in the direction in which sample tissue is intended to be collected, without agitating tissue on the opposite side of the textured scraping body 400.

Enlarged pockets 402 can also facilitate flexing of shaft 408. Troughs 412 can comprise a thinned-down portion of shaft 408 to allow shaft to flex at troughs 412, thereby allowing scraping edge 404 and scraping edge 406 to tilt outward to better engage tissue. In examples, troughs 412 can be positioned at locations of about fifty-percent of the diameter of shaft 408 to about eight-percent of the diameter of shaft 408, leaving shaft 408 to have a thickness of about fifty-percent to about twenty-percent of the diameter at the location of troughs 412.

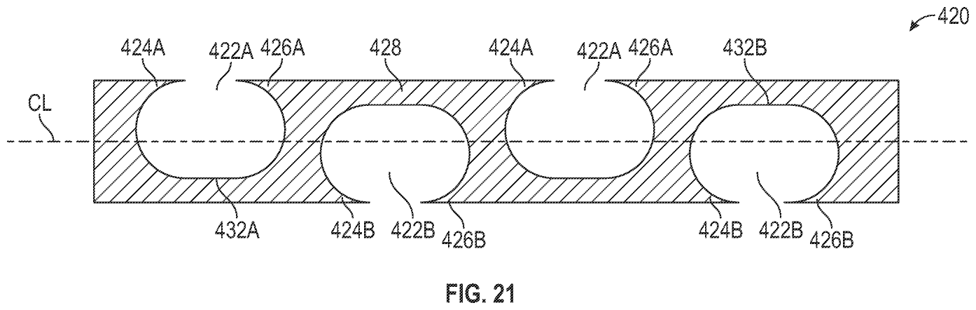

FIG. 21 is a schematic side view of a textured scraping body 420 for a ductal sampling device of the present disclosure comprising enlarged pockets 422A forming scraping edges 424A and 426A and enlarged pockets 422B forming scraping edges 424B and 426B arranged along shaft 428 in an alternating pattern to face in opposite directions.

In cross-section, enlarged pockets 422A and 422B can be similar in shape and function as scraping edges 219 of FIG. 10. However, rather than comprising annular bodies surrounding centerline CL, enlarged pockets 422A and 422B can extend straight through shaft 428 to form an axial passage having an inlet and outlet. Enlarged pockets 422A can comprise troughs 432A that are located on the opposite side of centerline CL than scraping edges 424A and 426A, and enlarged pockets 422B can comprise troughs 432B that are located on the opposite side of centerline CL than scraping edges 424B and 426B. As such, enlarged pockets 422A and 422B can be larger in volume than pockets 220 of FIG. 10.

Textured scraping body 420 can function similarly to functional scraping body 400 of FIG. 20A, except textured scraping body 420 can perform scraping in two radial directions, but still less than a three-hundred-sixty-degree arc. Troughs 432A and 432B can be located in similar positions as trough 312 of FIG. 2A and can have the same benefits of increasing flexibility.

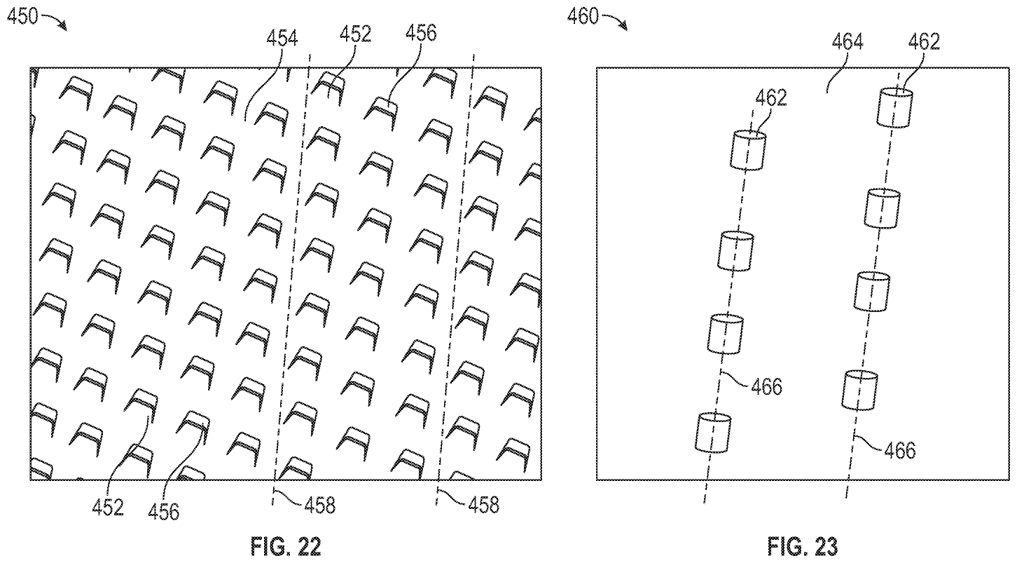

FIG. 22 is a perspective view of textured scraping body 450 of the present disclosure comprising outwardly facing scraping tabs 452. Outwardly facing scraping tabs 452 can be formed from sheet 454, such as by cutting an arc or c-shaped cut-out in sheet 454. Outwardly facing scraping tabs 452 can be bent outward from sheet 454 to form openings 456. In examples, outwardly facing scraping tabs 452 can be configured to function similar to a cheese grater or lemon zester. Outwardly facing scraping tabs 452 can be arranged in columns to form spaces extending along axes 458. Thus, outwardly facing scraping tabs 452 can be arranged in a plurality of rows and columns to form an array of textured scraping bodies. The edges of tabs 452 can be sharp or rough to cause separation of tissue. The separated tissue can be forced or otherwise moved into openings 456 to collect the tissue for analysis. Sheet 454 can be rolled into a cylindrical shaft shape to form a portion of a ductal sampling device of the present disclosure.

FIG. 23 is a perspective view of sheath 460 for use with textured scraping body 450 of FIG. 22 having standoffs 462 to provide separation from outwardly facing scraping tabs 452. Standoffs 462 can extend from sheet 464. Standoffs 462 can be arranged in columns extending along axes 466. Standoffs 562 can be sized to fit between columns of outwardly facing tabs 452 of FIG. 22. Thus, columns of standoffs 562 can slide along sheet 454 at axes 458 to prevent outwardly facing tabs 452 from sticking or binding on sheath 460. Standoffs 462 can comprise bodies attached to sheet 464 or can comprise bulges or bumps formed into sheet 464, such as rounded dimples. In examples, sheath 460 can comprise sheath 406 of FIG. 6.

Could make sheath/grater that only goes in one direction in cholangioscope. The teeth could face proximally so you can extend grater into sheath, but then you pull both out and cut off sheath. Push grater to protrude distally, manipulate back and forth, remove choalangioscope and shaver together with shafter still sticking out. When the two are out of duodenoscope, advance shaver a little more and cut end of shaver off. You can then pull rest of shaver device without teeth back through cholangioscope without damaging it. This might not work b/c you might damage the duodenoscope.

FIG. 24 is a schematic cross-sectional view of ductal sampling device 500 of the present disclosure connected to fluid system 502. Ductal sampling device 500 can comprise tip 504, textured scraping body 506, shaft 508, handle 510 and internal passage 512. Fluid system 502 can comprise injector 514, pump 516, fluid line 518 and coupler 520.

Ductal sampling device 500 can be configured similarly as ductal sampling device 200 of FIG. 10. Internal passage 512 of ductal sampling device 500 can extend from textured scraping body 506 through shaft 508 and through handle 510.

Fluid line 518 can comprise one or more lengths of flexible tubing. Coupler 520 can comprise a luer connector. Injector 514 and pump 516 can comprise an injection system to move fluid into and out of the ductal sampling devices of the present disclosure. In examples, injector 514 can comprise a syringe. In examples, pump 516 can comprise a fluid pump or compressor.

One or both of injector 514 and pump 516 can be used to introduce a fluid, such as saline, irrigation fluid, lavage fluid or another fluid, into ductal sampling device 500, either while the ductal sampling device is inserted into anatomy or afterwards. In examples, one or both of injector 514 and pump 516 can be used to push collected biological material out of the ductal sampling device using a fluid so the biological material can be collected and analyzed. In examples, one or both of injector 514 and pump 516 can be used to suction or vacuum collected biological material out of the ductal sampling device.

### Examples of How To Operate Ductal Sampling Device

1) Insert the device to working channel of Cholangioscope. The device can be advanced to the tip of the scope. Once the device reach the distal end, a physician or user can see the distal tip of the device by seeing the image from Cholangioscope.

2) Once target anatomy is visualized, the outer sheath actuator can be pulled back to expose the inner sheath.

3) The physician or user can grip the catheter and move the entire device in and out. By moving the device in and out several times, the texture sampling portion can agitate the region of interest and capture the tissue samples, similar to a cheese grater.

4) Once a tissue sample is obtained, the outer sheath actuator can be moved forward to cover the inner sheath's textured sampling portion.

5) Remove the entire device, including the outer sheath and the inner catheter.

6) Clip textured portion of inner sheath with outer sheath still covering it and place in cytological specimen container for examination by cytologist.

FIG. 10 is block diagram illustrating examples of method 900 of collecting biological matter from a patient using the tissue retrieval devices of the present disclosure, such as those including textured scraping body 210. Method 900 can encompass the use of ductal sampling device 200 of FIGS. 6-9, as well as other instruments. Method 900 can additionally be used with the devices and systems of FIGS. 10-24.

At step 902, an endoscope, such as a duodenoscope, can be inserted into and navigated through anatomy of a patient. For example, endoscope 14 (FIG. 1) can utilize native imaging capabilities to guide insertion section 28 through anatomic ducts of the patient. Insertion section 28 can be bent or curved using control knob 38 to facilitate turning of endoscope 14.

At step 904, an auxiliary scope can be inserted into the endoscope to access anatomy located further in the duct. For example, auxiliary scope 134 (FIG. 5) can be inserted into central lumen 62 (FIG. 3C) or lumen 132 (FIG. 5) to reach another anatomic duct intersecting the anatomic duct reached by endoscope 14. Elevator 54 (FIG. 3C) can be used to bend or turn auxiliary scope 134.

At step 906, a tissue retrieval device or ductal sampling device can be inserted into the auxiliary scope to reach target tissue distal of the auxiliary scope. The target tissue can comprise tissue that is potentially diseased or otherwise indicative of a diseased condition of the patient. For example, ductal sampling device 200 (FIG. 6) can be inserted such that textured scraping body 210 extends beyond the distal end of auxiliary scope 134.

At step 908, the tissue collection device can be navigated to the location of target tissue within the patient. For example, ductal sampling device 200 can be navigated through an anatomic duct to target tissue. The target tissue can comprise tissue that is potentially diseased or otherwise indicative of a diseased condition of the patient.

At step 910, a viewing device on the auxiliary scope can be activated in order to view biological matter of the patient. For example, imaging unit 110 (FIG. 5) can be activated to view anatomy in field of the view of camera lens 137 (FIG. 5).

At step 912, target tissue can be viewed using an imaging unit and a video display monitor. For example, imaging unit 110 can use objective lens 80 to display target tissue on output unit 18. Objective lens 80 can view the target tissue and textured scraping body 210 at the same time or simultaneously. Light from a light source can be used to illuminate the target tissue. For example, light from illumination lens 78, as generated by lighting unit 112, can be directed upon the target tissue.

At step 914, outer sheath 204 surrounding textured scraping body 210 can be retracted. For example, a user can slide actuator 214 proximally toward handpiece 206 to expose textured scraping body 210. Additionally, retraction of outer sheath 204 can release pre-curvature of scraping body 210, as described herein.

At step 915, a tissue collection device can be pushed, pressed or otherwise brought into pressurized contact with the target tissue. Thus, ductal sampling device 200 can be reciprocated axially, or rotated, to cause textured scraping body 210 to slice, punch or shave, etc. one or more pieces of tissue away from the anatomy of the patient. Additionally, at step 915, textured scraping body 210 can be bent, bowed or curved to facilitate engagement of cutting teeth 219 with tissue. For example, activation wire 340 can be pulled to induce bending or bowing, as shown in FIGS. 19B and 19C.

At step 916, sample tissue or biological matter separated or collected from the patient at step 914 can be stored within a space inside the tissue collection device. For example, as ductal sampling device 200 is manipulated back-and-forth, or rotated, separated sample tissue can be positioned within pockets 220. Furthermore, biological matter can be stored in internal passage 230 (FIG. 10).

At step 918, outer sheath 204 can be extended to cover textured scraping body 210 and collected tissue. For example, a user can slide actuator 214 distally away from handpiece 206 to cover textured scraping body 210 with outer sheath 204.

At step 920, the tissue collection device can be removed from the patient, such as by removal from the auxiliary scope, which can be left in place inside the anatomy. Outer sheath 204 can provide a safeguard to ensure removal of ductal sampling device 200 without inadvertently cutting anatomy of the patient.

At step 922, the collected sample tissue can be removed from the tissue collection device. In examples, textured scraping body 210 can be separated from shaft 208 to remove a sample tissue for analysis, etc. For example, shaft 208 can be cut to separate textured scraping body 210 along with a portion of outer sheath 204 to transport the collected biological matter without disturbance. Similarly, break-away section 240 (FIG. 12) can be broken to remove textured scraping body 210. Additionally, scraper 260 (FIG. 14 can be used to push biological matter out of textured scraping body 210. Furthermore, fluid or suction can be used to push collected biological mater from textured scraping body 210, as described with reference to FIG. 24.

At step 924, the auxiliary scope can be removed from the endoscope. For example, auxiliary scope 134 (FIG. 5) can be withdrawn from endoscope 14 (FIG. 1).

At step 926, the endoscope can be removed from the patient. For example, endoscope 100 (FIG. 5) can be withdrawn from duodenum D. The patient can thereafter be appropriately closed up or prepared for completion of the procedure.

As such, method 900 illustrates examples of a method of collecting biological matter from internal passages of a patient in large enough quantities, e.g., by using a textured scraping body having internal storage, to eliminate or reduce insertion and removal of surgical devices from the patient.

### Examples

Example 1 is a ductal sampling device comprising: an elongate shaft comprising: a proximal end portion; and a distal end portion comprising a textured scraping body; and a sheath configured to slide over the elongate shaft to alternatively cover and cover the textured scraping body.

In Example 2, the subject matter of Example 1 optionally includes an endoscope comprising: an insertion section having a working channel in which the elongate shaft can be disposed; and an imaging device configured to view the textured scraping body when extended from the working channel.

In Example 3, the subject matter of Example 2 optionally includes a duodenoscope configured to receive the endoscope.

In Example 4, the subject matter of any one or more of Examples 1-3 optionally include wherein the elongate shaft further comprises an atraumatic tip disposed distal of the textured scraping body, wherein the atraumatic tip comprises an elongate flexible cap.

In Example 5, the subject matter of any one or more of Examples 1˜4 optionally include wherein the elongate shaft further comprises a handpiece connected to the proximal end portion.

In Example 6, the subject matter of any one or more of Examples 1-5 optionally include wherein the sheath comprises an actuator to facilitate sliding of the sheath along the elongate shaft.

In Example 7, the subject matter of Example 6 optionally includes wherein the actuator comprises a knob.

In Example 8, the subject matter of any one or more of Examples 1-7 optionally include wherein the textured scraping body comprises: a plurality of teeth; and a plurality of pockets interspersed with the plurality of teeth.

In Example 9, the subject matter of any one or more of Examples 1-8 optionally include wherein the textured scraping body comprises: a plurality of rows of cutting elements configured to slice or abrade tissue; and internal storage space configured to receive tissue from the plurality of rows of cutting elements.

In Example 10, the subject matter of Example 9 optionally includes wherein: the internal storage space comprises a plurality of pockets recessed within the elongate shaft; and each of the cutting elements comprises an axially projecting edge that overhangs one of the plurality of pockets.

In Example 11, the subject matter of Example 10 optionally includes wherein each of the cutting elements comprises an axially projecting blunt end.

In Example 12, the subject matter of any one or more of Examples 10-11 optionally include wherein the internal storage space further comprises: a plurality of radially extending passages extending from each of the plurality of pockets; and a lumen extending axially through the elongate shaft.

In Example 13, the subject matter of any one or more of Examples 10-12 optionally include wherein each of the plurality of pockets comprises an annular pocket circumscribing the elongate shaft.

In Example 14, the subject matter of any one or more of Examples 10-13 optionally include wherein each of the plurality of pockets comprises an axial pocket extending radially across the elongate shaft.

In Example 15, the subject matter of any one or more of Examples 10-14 optionally include wherein: the internal storage space comprises a lumen extending axially through the elongate shaft; and each of the cutting elements comprises a tab projecting at an angle outward from the elongate shaft to form: a cutting edge; and an opening in the elongate shaft connecting to the lumen.

In Example 16, the subject matter of any one or more of Examples 9-15 optionally include a longitudinal lumen extending within the elongate shaft to define at least a portion of the internal storage space; a slit extending along at least a portion of the elongate shaft; a paddle configured to slide along the longitudinal lumen; and a handle extending from the paddle through the slit.

In Example 17, the subject matter of any one or more of Examples 9-16 optionally include a longitudinal lumen extending within the elongate shaft to define at least a portion of the internal storage space; and a fluid injection or suction system connected to the longitudinal lumen.

In Example 18, the subject matter of any one or more of Examples 1-17 optionally include wherein the textured scraping body comprises a plurality of flexible linkages formed in the elongate shaft.

In Example 19, the subject matter of Example 18 optionally includes wherein each of the plurality of flexible linkages comprises a trapezoidal shaped body positioned within an opposing trapezoidal shaped cut-out.

In Example 20, the subject matter of any one or more of Examples 1-19 optionally include a pull wire attached to the distal end portion to deflect the textured scraping body.

In Example 21, the subject matter of Example 20 optionally includes wherein the pull wire is configured to induce bending of the textured scraping body.

In Example 22, the subject matter of any one or more of Examples 20-21 optionally include wherein the pull wire is configured to induce bulging of the textured scraping body.

In Example 23, the subject matter of any one or more of Examples 1-22 optionally include wherein the elongate shaft includes pre-curvature at the textured scraping body.

In Example 24, the subject matter of any one or more of Examples 1-23 optionally include a break-away section located between the elongate shaft and the textured scraping body.

Example 25 is a method of collecting biological matter using a ductal sampling device, the method comprising: inserting the ductal sampling device into anatomy of a patient; guiding a textured scraping body of the ductal sampling device to a target tissue; grating the textured scraping body against the target tissue; and collecting biological matter from the target tissue with the textured scraping body.

In Example 26, the subject matter of Example 25 optionally includes viewing the target tissue and the textured scraping body with an endoscope from which the ductal sampling device extends.

In Example 27, the subject matter of any one or more of Examples 25-26 optionally include wherein grating the textured scraping body against the target tissue comprises slicing portions of the target tissue using teeth of the textured scraping body.

In Example 28, the subject matter of any one or more of Examples 25-27 optionally include wherein collecting biological matter from the target tissue with the textured scraping body comprises capturing portions of the target tissue within pockets of the textured scraping body.

In Example 29, the subject matter of any one or more of Examples 25-28 optionally include retracting a sheath to expose the textured scraping body to the target tissue to performing grating of the target tissue.

In Example 30, the subject matter of Example 29 optionally includes extending the sheath to trap grated target tissue between the textured scraping body and the sheath.

In Example 31, the subject matter of any one or more of Examples 25-30 optionally include engaging an atraumatic distal tip of the ductal sampling device with a duct wall proximate the target tissue.

In Example 32, the subject matter of any one or more of Examples 25-31 optionally include pulling an activator to induce bending of the textured scraping body to expose a scraping edge.

In Example 33, the subject matter of Example 32 optionally includes wherein pulling the activator comprises pulling a pull wire extending within an insertion shaft connected to the textured scraping body or pulling an insertion sheath configured to surround the textured scraping body.

In Example 34, the subject matter of any one or more of Examples 25-33 optionally include activating a fluid system to remove collected biological matter from the textured scraping body.

In Example 35, the subject matter of any one or more of Examples 25-34 optionally include severing the textured scraping body from an insertion shaft to facilitate removal of collected biological matter from the textured scraping body.

In Example 36, the subject matter of any one or more of Examples 25-35 optionally include flexing the textured scraping body along a plurality of interconnected scraping linkages.

Each of these non-limiting examples can stand on its own, or can be combined in various permutations or combinations with one or more of the other examples.

### Notes

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventor also contemplates examples in which only those elements shown or described are provided. Moreover, the present inventor also contemplates examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A ductal sampling device comprising:
an elongate shaft comprising:
a proximal end portion; and
a distal end portion comprising a textured scraping body; and
a sheath configured to slide over the elongate shaft to alternatively cover and cover the textured scraping body.

2. The ductal sampling device of claim 1, further comprising:
an endoscope comprising:
an insertion section having a working channel in which the elongate shaft can be disposed; and
an imaging device configured to view the textured scraping body when extended from the working channel; and
a duodenoscope configured to receive the endoscope.

3. The ductal sampling device of claim 1, wherein:
the elongate shaft further comprises:
an atraumatic tip disposed distal of the textured scraping body, wherein the atraumatic tip comprises an elongate flexible cap; and
a handpiece connected to the proximal end portion; and
the sheath comprises an actuator to facilitate sliding of the sheath along the elongate shaft, wherein the actuator comprises a knob.

4. The ductal sampling device of claim 1, wherein the textured scraping body comprises:
a plurality of rows of cutting elements configured to slice or abrade tissue; and
internal storage space configured to receive tissue from the plurality of rows of cutting elements.

5. The ductal sampling device of claim 4, wherein:
the internal storage space comprises:
a plurality of pockets recessed within the elongate shaft, each of the plurality of pockets comprises an annular pocket circumscribing the elongate shaft;
a plurality of radially extending passages extending from each of the plurality of pockets; and
a lumen extending axially through the elongate shaft connecting to the plurality of radially extending passages.

6. The ductal sampling device of claim 5, wherein:
each of the cutting elements comprises an axially projecting edge that overhangs one of the plurality of pockets, each axially projecting edge comprises:
a tab projecting at an angle outward from the elongate shaft to form:
a cutting edge; and
an opening in the elongate shaft connecting to the lumen.

7. The ductal sampling device of claim 4, further comprising:
a longitudinal lumen extending within the elongate shaft to define at least a portion of the internal storage space;
a slit extending along at least a portion of the elongate shaft;
a paddle configured to slide along the longitudinal lumen; and
a handle extending from the paddle through the slit.

8. The ductal sampling device of claim 4, further comprising:
a longitudinal lumen extending within the elongate shaft to define at least a portion of the internal storage space; and
a fluid injection or suction system connected to the longitudinal lumen.

9. The ductal sampling device of claim 1, wherein the textured scraping body comprises a plurality of flexible linkages formed in the elongate shaft, wherein each of the plurality of flexible linkages comprises a shaped body positioned within an opposing shaped cut-out.

10. The ductal sampling device of claim 1, further comprising a pull wire attached to the distal end portion to deflect the textured scraping body, wherein the pull wire is configured to induce bending or bulging of the textured scraping body.

11. The ductal sampling device of claim 1, wherein the elongate shaft includes pre-curvature at the textured scraping body.

12. The ductal sampling device of claim 1, further comprising a break-away section located between the elongate shaft and the textured scraping body.

13. A method of collecting biological matter using a ductal sampling device, the method comprising:
inserting the ductal sampling device into anatomy of a patient;
guiding a textured scraping body of the ductal sampling device to a target tissue;
grating the textured scraping body against the target tissue; and
collecting biological matter from the target tissue with the textured scraping body.

14. The method of claim 13, further comprising viewing the target tissue and the textured scraping body with an endoscope from which the ductal sampling device extends.

15. The method of claim 13, wherein:
grating the textured scraping body against the target tissue comprises slicing portions of the target tissue using teeth of the textured scraping body; and
collecting biological matter from the target tissue with the textured scraping body comprises capturing portions of the target tissue within pockets of the textured scraping body.

16. The method of claim 13, further comprising:
retracting a sheath to expose the textured scraping body to the target tissue to performing grating of the target tissue; and
extending the sheath to trap grated target tissue between the textured scraping body and the sheath.

17. The method of claim 13, further comprising pulling an activator to induce bending of the textured scraping body to expose a scraping edge, wherein pulling the activator comprises pulling a pull wire extending within an insertion shaft connected to the textured scraping body or pulling an insertion sheath configured to surround the textured scraping body.

18. The method of claim 13, further comprising activating a fluid system to remove collected biological matter from the textured scraping body.

19. The method of claim 13, further comprising severing the textured scraping body from an insertion shaft to facilitate removal of collected biological matter from the textured scraping body.

20. The method of claim 13, further comprising flexing the textured scraping body along a plurality of interconnected scraping linkages.
